(19) United States
(12) Patent Application Publication
(10) Pub. No.: US 2026/0078368 A1
(43) Pub. Date: Mar. 19, 2026

(54) METHODS AND COMPOSITIONS FOR TREATING PANCREATIC CANCER

(71) Applicants: Sanford Burnham Prebys Medical Discovery Institute, La Jolla, CA (US); Cedars-Sinai Medical Center, Los Angeles, CA (US)

(72) Inventors: Randal J. KAUFMAN, San Diego, CA (US); Jing YONG, San Diego, CA (US); Stephen PANDOL, Los Angeles, CA (US)

(21) Appl. No.: 18/879,053

(22) PCT Filed: Jun. 27, 2023
(86) PCT No.: PCT/US2023/069201

§ 371 Date: Dec. 26, 2024

Related U.S. Application Data
(60) Provisional application No. 63/356,441, filed on Jun. 28, 2022.

(51) Int. Cl. C12N 15/113; A61P 35/00

(52) U.S. Cl. CPC C12N 15/113; A61P 35/00; C12N 2310/11

(57) ABSTRACT

Methods and compositions related to treating pancreatic cancer are described.

### CROSS-REFERENCE

This application is a national stage application of International Application No. PCT/US2023/069201, filed Jun. 27, 2023, which claims the benefit of U.S. Provisional Application No. 63/356,441, filed Jun. 28, 2022, each of which is incorporated herein by reference in its entirety.

### STATEMENT AS TO FEDERALLY SPONSORED RESEARCH

This invention was made with government support under P30 CA030199 awarded by the National Institutes of Health. The government has certain rights in the invention.

### INCORPORATION BY REFERENCE OF SEQUENCE LISTING

The present application contains a Sequence Listing which has been submitted in electronic format and is hereby incorporated by reference in its entirety. Said Sequence Listing is provided as a file entitled 42256-611_831_SL.xml, created Oct. 10, 2025, and is 21,347 bytes in size.

### BACKGROUND

Type 2 diabetes (T2DM) is a metabolic disorder that poses a severe health challenge for modern society as it is estimated by the United States' Centers for Disease Control and Prevention that thirty million Americans are affected by this condition. Enhanced insulin synthesis is associated with proinsulin misfolding and endoplasmic reticulum (ER) stress.

Type 2 diabetes (T2DM) is a risk factor for Pancreatic Ductal Adenocarcinoma (PDAC). PDAC remains one of the most lethal human solid tumors, despite great efforts in improving therapeutics over the past few decades. There is currently no effective treatment for PDAC.

### SUMMARY OF THE DISCLOSURE

The present disclosure provides methods and compositions for treating pancreatic cancer, e.g., associated with Type 2 diabetes (T2D). In one aspect, the present disclosure provides a method of treating pancreatic cancer, e.g., associated with T2D, in a subject in need thereof, comprising: selectively inhibiting CCAAT/enhancer-binding protein (C/EBP) homologous protein (CHOP) in pancreatic β cells, e.g., by administering to the subject a composition comprising: (a) a CHOP inhibiting moiety, and (b) a pancreatic β cell targeting moiety. In some embodiments, the method can be used for treating PDAC. In some embodiments, the method for treating pancreatic cancer and/or treating PDAC comprises inhibiting CHOP selectively in pancreatic β cells. In some embodiments, the method comprises treating pancreatic cancer. In some embodiments, the method comprises treating PDAC. In some embodiments, the subject is a mammal. In some embodiments, the subject is a human. In some embodiments, the instant disclosure provides methods and compositions that may prevent aging associated non-alcoholic fatty liver disease (NAFLD), or at least a symptom or a manifestation associated thereof.

In another aspect, the present disclosure provides a method of regulating C/EBP homologous protein (CHOP) in pancreatic β cells. In some embodiments, the method comprises administering a nucleic acid composition comprising: (a) a CHOP inhibiting moiety, and (b) a targeting moiety that directs the CHOP inhibiting moiety to a target in a pancreatic cell. In some embodiments, regulating C/EBP homologous protein (CHOP) in pancreatic β cells ameliorate or treats pancreatic cancer.

In some embodiments, a CHOP inhibiting moiety is a nucleic acid. In some embodiments the CHOP inhibiting moiety and the pancreatic β cell targeting moiety are operably linked. In some embodiments, the nucleic acid is an RNA. In some embodiments, the nucleic acid is an inhibitory RNA. In some embodiments, the nucleic acid is an antisense oligomeric RNA. In some embodiments, the nucleic acid is an iRNA.

In some embodiments, the pancreatic β cell targeting moiety is a peptide. In some embodiments, the peptide is internalized by a pancreatic cell. In some embodiments, the peptide is glucagon-like peptide 1 (GLP-1), or a fragment thereof.

In some embodiments, the CHOP inhibiting moiety is a nucleic acid editing moiety. In some embodiments, the nucleic acid editing moiety is a genomic DNA editing moiety. In some embodiments, the nucleic acid editing moiety comprises a nuclease. In some embodiments, the nucleic acid editing moiety comprises a recombinase. In some embodiments, the pancreatic β cell targeting moiety comprises a guiding nucleic acid sequence.

In some embodiments, the CHOP inhibiting moiety and/or the pancreatic β cell targeting moiety is inducible by an inducer. In some embodiments, the inducer can be administered ex vivo. In some embodiments, the targeting moiety is inducible by an inducer. In some embodiments, the inducer is administered ex vivo. In some embodiments, the inducer is tamoxifen.

In some embodiments, wherein the subject is a mammal. In some embodiments, the subject is a human. In some embodiments, the subject is a non-human mammal. In some embodiments the administering comprises administering to the subject systemically. In some embodiments, the administering reduces or alleviates pancreatic β cell stress. In some embodiments, the administering reduces total pancreatic insulin content.

In one aspect, the present disclosure provides a nucleic acid composition comprising a nucleic acid sequence capable of suppressing a CHOP gene expression, operably linked to a peptide. In some embodiments, the peptide is conjugated to the nucleic acid sequence. In some embodiments, the nucleic acid composition comprises a nucleic acid construct comprising: (a) an antisense oligomeric (ASO) sequence, and (b) a GLP-1 peptide or a fragment thereof. In some embodiments, the nucleic acid construct further comprises a linker.

In some embodiments, the nucleic acid sequence capable of suppressing a CHOP expression is an RNA. In some embodiments, the RNA is an inhibitory RNA. In some embodiments, the RNA is an antisense oligomeric RNA. In some embodiments, the RNA is an iRNA. In some embodiments, nucleic acid composition is targetable to a pancreatic cell. In some embodiments, nucleic acid composition is targetable to a pancreatic β cell.

In some embodiments, the peptide is glucagon-like peptide 1 (GLP-1), or a fragment thereof.

In some embodiments, the nucleic acid composition further comprises a linker. In some embodiments, the linker is a synthetic linker. In some embodiments, the linker may be a chemical linker. In some embodiments, the linker may be a short peptide linker. In some embodiments, the linker physically connects the antisense oligomeric sequence, and the GLP-1 peptide.

The linker can be a chemical linker. The linker can be a synthetic linker. The linker may be able to crosslink the nucleic acid sequence and the peptide.

In some embodiments, the nucleic acid composition comprises a nucleic acid sequence-a linker-a GLP1 peptide.

In some embodiments the nucleic acid composition further comprises a delivery vehicle. In some embodiments, the delivery vehicle comprises a lipid component. In some embodiment the lipid is in the form of a liposome. In some embodiments, the delivery vehicle comprises a lipid, such as a cationic lipid. In some embodiments, the delivery vehicle comprises or a vector, such as a viral vector or a nucleic acid construct comprising the nucleic acid sequence. In some embodiments, the nucleic acid composition comprises a vector.

In some embodiments the present disclosure provides a cell comprising a composition described herein, or a part thereof.

In some embodiments, the nucleic acid composition can be used for preparing a therapeutic for treating diabetes and pancreatic cancer. In some embodiments, the nucleic acid composition that can be used for preparing the therapeutic for treating diabetes and pancreatic cancer is inside a cell. In some embodiments the cell is a pancreatic β cell. In some embodiments, the pancreatic cancer is PDAC.

In some embodiments, the present disclosure provides a pharmaceutical composition, comprising the nucleic acid described herein or a part thereof, and a pharmaceutically acceptable carrier. The nucleic acid may be comprised in a cell. In some embodiments, the nucleic acid described herein, or a part thereof may be comprised in a vector. In some embodiments, the pharmaceutical composition, comprising the nucleic acid comprises the vector.

In some embodiments, the present disclosure provides a method of treating hyperinsulinemia and hyperglycemia in a subject comprising, administering a treatment comprising a pharmaceutical composition wherein the pharmaceutical composition comprises: (a) a CHOP inhibiting moiety, and (b) a pancreatic B cell targeting moiety, and wherein the administering of the treatment inhibits C/EBP homologous protein (CHOP) in pancreatic β cell and further, wherein the inhibition of CHOP alleviates hyperinsulinemia and hyperglycemia-associated disorder.

In some embodiments, the method comprises, administering to a subject a treatment comprising a pharmaceutical composition wherein the pharmaceutical composition comprises: (a) a CHOP inhibiting moiety, and (b) a pancreatic B cell targeting moiety, where the hyperinsulinemia and hyperglycemia-associated disorders comprise dysregulated insulin secretion disorders. In some embodiments, the method comprises treating fatty liver disease in diabetes. In some embodiments, the method can be used for treating endoplasmic reticulum (ER) stress. In some embodiments, the method can be used for treating fatty liver disease in diabetes. In some embodiments, the method can be used for treating glucose intolerance. In some embodiments, the method can be used for treating insulin resistance. In some embodiments, the method can be used for treating pancreatic steatosis. In some embodiments, the method can be used for treating liver steatosis.

In some embodiments, provided herein is a method of inhibiting pancreatic adenocarcinoma (PDAC) growth in a subject comprising, administering a treatment comprising a pharmaceutical composition wherein the pharmaceutical composition comprises: (a) a CHOP inhibiting moiety, and (b) a pancreatic B cell targeting moiety, and where the administering of the treatment inhibits C/EBP homologous protein (CHOP) in pancreatic B cell and further, where the inhibition of CHOP activates effector T cells in a subject with an obesity-induced pathophysiology. In some embodiments, provided herein is the method of administering to a subject a treatment comprising inhibiting a tumor. In some embodiments, provided herein is a method of administering to a subject a treatment comprising inhibiting the tumor comprising PDAC. In some embodiments, provided herein is the method of administering a treatment comprising inhibiting a tumor comprising metastasis to the liver. In some embodiments, provided herein is a method of inhibiting pancreatic adenocarcinoma (PDAC) growth in a subject, where the subject with the obesity-induced pathophysiology comprises an obesity that is associated acceleration of primary tumorigenesis. In some embodiments, provided herein is a method of inhibiting pancreatic adenocarcinoma (PDAC) growth comprising, administering to a subject a treatment comprising a pharmaceutical composition wherein the pharmaceutical composition comprises: (a) a CHOP inhibiting moiety, and (b) a pancreatic B cell targeting moiety, and where the administering of the treatment inhibits C/EBP homologous protein (CHOP) in pancreatic B cell and further, where the inhibition of CHOP alleviates effects of β cell dysfunction. In some embodiments, provided herein is the method of inhibiting pancreatic adenocarcinoma (PDAC) growth in a subject, wherein the effects of β cell dysfunction comprise pancreatic cancer metastasis to the liver. In some embodiments, is a method of inhibiting pancreatic adenocarcinoma (PDAC) growth in a subject, wherein the effects of β cell dysfunction comprise initiation and progression of PDAC.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1. Illustrates an exemplary schematic of the development of pancreatic ductal adenocarcinoma (PDAC) and metastasis via hyperinsulinemia by obesity and/or diabetes. Shown is the mechanism of the studies disclosed herein: High levels of local and circulating insulin (and IGF1) activate growth signaling promotes survival and pro-fibroinflammatory responses in cancer and stromal cells (including fibroblast-type cells and immune cells), which are all conducive to the development of primary tumors and metastases. In addition, patients with type-2 diabetes mellitus (T2DM) exhibit significant fibrosis in areas adjacent to islets; leading to perturbed and inflamed islets which may promote excessive insulin signaling and tumor development in diabetic patients with oncogenic Kras mutations. Similarly, fatty liver contributes to development of liver metastases.

FIG. 2A-D. Illustrates an effect of CCAAT/enhancer-binding protein (C/EBP) homologous protein (CHOP) antisense oligomer (CHOP ASO) treatment on glucose metabolism in KC mice that were fed a control diet (CD) or high-fat diet (HF). Glucose tolerance tests were performed after four weeks of control ASO or CHOP ASO treatment in mice that received the CD or HF. As seen in FIG. 2A and FIG. 2C, KC mice fed CD or HF and treated with control ASO showed similar fasting blood glucose levels, but glucose levels remained slightly elevated in the HF-fed mice during the glucose challenge (FIG. 2A). CHOP ASO treatment resulted in improved glucose tolerance (FIG. 2B), with CHOP ASO treated mice displaying lower area under the curve (AUC) than controls especially in HF-fed KC mice. KC mice that were treated with HF diet showed considerable weight gain in comparison to KC mice fed with CD (FIG. 2D). These data suggest that CHOP silencing in β cells improves glucose tolerance in HF fed KC mice. Data are presented as means±SEM, N=3 to 6 mice per group.

FIG. 3A-B. Illustrates an effect of CHOP ASO treatment on Pancreatic Intraepithelial Neoplasia (PanINs) and PDAC progression in KC mice fed control (CD) or high-fat diet (HF). In FIG. 3A, Hematoxylin and eosin stain (H & E) diagrams show pancreas histology in 15-week-old mice that were treated with control or CHOP-ASO as indicated. (Areas with intra epithelial neoplasia (PanIN) or PDAC (top, middle diagrams only) are indicated by black arrows, while normal parenchyma is depicted by white arrows, scale bar, 500 μm. H&E staining in FIG. 3B, was used to show liver histology from the same mice seen in FIG. 3A. Fat droplets within hepatocytes are depicted by black arrows. Scale bar, 200 μm). Compared to CD-fed (FIG. 3A, left panel), obese KC mice fed HF and treated with control ASO displayed a marked transformation of the pancreas parenchyma: the landscape consists mainly of neoplastic ducts, cancer cells and extensive stromal areas enriched in immune cells and fibroblasts, normal parenchyma is rare in these tissues (FIG. 3A, central panel). As expected, HF-fed mice also displayed liver steatosis (FIG. 3B). Importantly, CHOP ASO treatment in HF-fed mice partially prevented the loss of normal parenchyma and reduced neoplastic duct numbers (FIG. 3A, right panel) and fatty liver (FIG. 3B). These data suggest that reduction of β cell CHOP levels is minimizing obesity-mediated acceleration of primary tumorigenesis.

FIG. 4A-D. Illustrates an exemplary effect of GLP1-CHOP ASO treatment on CHOP, also known as DNA Damage Inducible Transcript 3 (DDIT3), and insulin mRNA expression levels in pancreas of KC mice fed control diet (CD) or high fat (HF) diet. RNA levels of CHOP (FIG. 4A), insulin (FIG. 4B), the ductal marker (Sox9, FIG. 4C) and the stromal marker Collagen Type 1 alpha 1 (Col1a1, FIG. 4D) measured after 4 weeks of ASP treatment. Data from wild type mice (WT) fed CD or HF is included on the right bars of each graph as comparison. CHOP and insulin expression was significantly increased in pancreas of KC mice fed HF and treated with control ASO. Importantly, treatment with GLP1-CHOP ASO effectively reduced CHOP and insulin expression in HF-fed KC mice (FIG. 4A-B), and this effect was associated with significant decreases in mRNA levels of the ductal marker Sox9 and the stromal marker Col1a1 (a collagen chain type highly expressed in PDAC tumors), FIG. 4C-D. These data illustrate that the endocrine compartment of the pancreas modulates PDAC initiation and progression in obese mice through mechanisms involving abnormal local and systemic insulin production.

FIG. 5A-B. illustrates histological staining of spleen and liver tissues following injections with KPC cells. FIG. 5A depicts H & E staining of a spleen depicting tumor formation from injected KPC cells (scale bar=3 mm). FIG. 5B depicts H & E staining of a liver displaying metastatic tumor (scale bar=200 μm). The syngeneic orthotopic model of liver metastasis was established in the first year by intrasplenic injection of pancreatic KPC cells into CHOP βKO and control littermate mice with no CHOP deletion. Both groups of mice were morbidly sick 12d post KPC cell injection, due to rapid metastasis of KPC cells into liver, following the standard 1 million KPC cells transplant. Mice were sacrificed 12 days after receiving about 1 million KPC cells via intrasplenical injection. Metastatic tumors in the spleen (FIG. 5A) and in the liver (FIG. 5B) were evident in sacrificed mice.

FIG. 6A-C. Illustrates an exemplary dataset showing CHOP (FIG. 6A) levels in human PDAC tumors. DAPI cellular stain (FIG. 6B) and colocalized view of CHOP/DAPI (FIG. 6C show expected or normal cells/morphology. Human PDAC tumor tissue was obtained from patients undergoing surgical resection of pancreatic cancer (pancreaticoduodenectomy. CHOP expression was found in the nucleus of neoplastic cell.

FIG. 7 is a schematic of the protocol and timeline for mice with CHOP knockout in R cells and mice with wild-type CHOP in β cells. The timeline on the left indicates the time at which KPC cells are injected into mice. On the right is a summary of protocols ran thereafter; specifically, assessments on liver metastasis, glucose and insulin blood levels, and glycemic control.

FIG. 8. Illustrates an exemplary showing of the effect of CHOP depletion in CHOP β knockout (βKO) mice. Fewer liver metastases are present for CHOP β knockout (βKO) mice.

FIG. 9. The schematic on the left panel provides exemplary percentages of the genes implicated in PDAC mutations. The schematic on the right panel indicates the timepoints of treatment with GLP1-chop ASO or ASO control in KC mice, and the subsequent protocol, which entails PanIN evaluation, and assessments on glycemia control and liver histology.

FIG. 10. Illustrates an exemplary showing of liver metastasis foci in wildtype (WT) mice, as visualized by H & E staining. CHOP βWT show liver metastases at 4 weeks post splenic KPC mice injection. Metastasis measured in the bar graph shows the number of metastases per section in which CHOP βWT mice showed more metastasis per section than in the CHOP βKO mice.

FIG. 11. Illustrates an exemplary showing of the liver metastasis as depicted in FIG. 10 but showing an enlarged/focus area in FIG. 11. A histogram representing the number of metastases per section is shown on the right for CHOP βWT and CHOP βKO mice.

FIG. 12. Illustrates an exemplary schematic of the interplay between the exocrine and endocrine system in modulation. As disclosed herein, ER proteostasis in β cells restrains PDAC development and metastasis.

FIG. 13A-B. Illustrates an exemplary showing of the generation of BiP-3×Flag mice strain for both genetic constructs in FIG. 13A and FIG. 13B. Figures disclose “KDEL” as SEQ ID NO: 23.

FIG. 14A-C. Illustrates exemplary data showing that CHOP deletion in β cells reduces hepatomegaly and hepatic steatosis. FIG. 14A comprises exemplary photographs of the appearance of fresh liver (top) and pancreas organs (bottom) immediately after tissue dissection for all mice. FIG. 14B is a graph representative of liver triglycerides (TGs) in CHOP-deleted mice compared to WT and βHet littermate control mice (P<0.05). FIG. 14C are exemplary photographs of tissues from mice with CHOP βKO (Fe/Fe: Cre) (left) and mice with a floxed CHOP gene with exon 3 deleted by Cre/ERT recombinase (Δ/Δ:Cre) (right).

FIG. 15. is a schematic illustrating a model of the process by which CHOP depletion in pancreatic B cells promote insulin release and result in glycogen formation and lipogenesis stimulation in the liver.

FIG. 16. Illustrates an exemplary data showing that GLP1-CHOP ASO reduces fatty liver in two mouse models of non-alcohol fatty liver disease (NAFLD).

FIG. 17A-F comprises summative figures of mice glucose clamping procedures and the resulting collected data. FIG. 17A is an illustration of a mouse glucose clamping procedure. FIG. 17B is a schematic representation of the timeline of events for the glucose clamping procedure.

FIG. 17C is a line graph of measurements of arterial insulin concentrations (ng/ml) during the course of the mouse glucose clamping procedure. FIG. 17D is a line graph of measurements of arterial glucose concentrations (mg/dl) during the course of the mouse glucose clamping procedure. FIG. 17E is a line graph of measurements of arterial C-peptide concentrations (pM) during the course of the mouse glucose clamping procedure. FIG. 17F is a line graph of measurements of the infusion of radiolabeled glucose (Glucose Infusion Rate, or GIR, in mg/kg/min) during the course of the mouse glucose clamping procedure.

FIG. 18A-C comprises summative figures for the experiment determining the relationship between upregulation of the liver de novo lipogenesis (DNL) pathway and mouse insulin circulation. FIG. 18A is a histogram representing the differential expression of a subset of genes (x-axis), recorded as transcripts per million (TPM) for normal mouse liver tissue expressing wild-type CHOP in β cells (“Liver_WT” or “CHOP βHet”) and knocked-out CHOP in β cells (“Liver_KO” or “CHOP βKO”), and mouse tissue from PDAC tumors with wild-type CHOP (“Tumor_WT”) and knocked-out CHOP (“Tumor_KO”). FIG. 18B is a histogram of insulin concentration (pg/ml or pg/mg) in mice treated with diluent or tamoxifen (TAM) measured by Luminex assays. FIG. 18C is a histogram of glucose concentration (mg/dl) in male mice treated with diluent or tamoxifen on Day 21 measured by Luminex assays.

### DETAILED DESCRIPTION

The present disclosure is based on a finding that alleviating ER stress in β cells while maintaining optimal insulin secretion can be possible in living systems which may reduce or alleviate one or more aspects of β cell dysfunction and associated diseases, conditions, symptoms, disorders and the like. In some embodiments, conditions or disorders associated with β cell dysfunction comprises conditions such as, for example and without limitation, type-2 diabetes (T2D), T2D associated diseases, pancreatic cancer, obesity, pancreatic ductal adenocarcinoma (PDAC), dysregulation of insulin, among many other disorders. In some embodiments, T2D may be characterized by hyperglycemia, insulin resistance or hyperinsulinemia.

### Endoplasmic Reticulum Stress Response

Early-stage T2D can be characterized by insulin synthesis and enhanced secretion of insulin synthesis by a pancreatic β cell. This enhanced synthesis of insulin by the β cell can be associated with proinsulin misfolding which can lead to endoplasmic reticulum (ER) stress. Briefly, ER stress from enhanced synthesis of insulin by β cells perturbs protein folding capacity of the ER causing an accumulation of unfolded protein. ER stress triggers induction of transcriptional processes, for example, transcription of genes encoding chaperons and folding enzymes localized in the ER. Signaling and transcriptional induction of protein genes modulates ER stress and homeostasis, and this process is triggered and governed by the release of the unfolded protein response (UPR). In some embodiments, the sensing of the ER stress that triggers UPR may be mediated by Ire1p/Ern1p protein kinase, an ER transmembrane protein or other protein. Upon activation and release, UPR sends signals triggering proteolysis and/or activation of transcriptional factors which modulates ER stress. Subsequently, transcriptional factors can translocate from the ER membrane or subcellular localization into the nucleus to modulating transcription of ER stress-response genes.

### CCAAT/Enhancer-Binding Protein (C/EBP) Homologous Protein (CHOP) Activation

Signaling of ER stress can activate an increase in differential transcription and translation. When the ER is stressed, UPR can trigger activation of transcriptional factors to modulate ER stress. In some embodiments, transcriptional factors such as, for example, CCAAT/enhancer-binding protein (C/EBP) homologous protein (CHOP or CHOP, both used interchangeably hereinafter) can be activated when the ER undergoes stress such as, for example, during stress, signaling activities connected to the ER can lead to the release of UPR. In some embodiments, CHOP can be a target of the composition disclosed herein. In some embodiments, CHOP can be a target of the methods or kits disclosed herein. In some embodiments, CHOP can be a target of the pharmaceuticals or formulations disclosed herein. In some embodiments, CHOP can be a target of treatments disclosed herein.

### Inhibition of Pancreatic β Cell-Specific CHOP

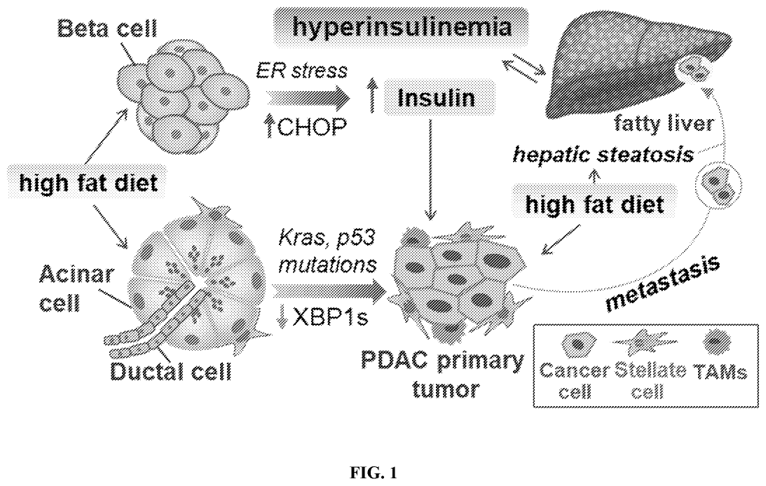

Type-2 diabetes mellitus (T2DM) and obesity may be risk factors for pancreatic ductal adenocarcinoma (PDAC) development and progression and may promote PDAC metastasis to the liver through multiple mechanisms (FIG. 1). Disclosed herein, in some embodiments, high levels of insulin synthesis and secretion by pancreatic β cells accelerates the rate of proinsulin misfolding and cause ER stress leading to upregulated transcription factor CHOP in β cells of the pancreas. In some embodiments, CHOP expression may promote insulin production in pancreatic β cells. In some embodiments, disclosed herein the CHOP signaling pathway positively regulates fatty liver formation. In some embodiments, deletion of pancreatic β cell-specific CHOP normalizes aberrant insulin and prevents hepatic steatosis when the subject with aberrant insulin secretion may be consuming high-fat diets (HF) or when the subject may not be consuming HF diets.

In some embodiments, inhibition of CHOP in pancreatic β cells may provide therapeutic benefits, such as for example, inhibiting CHOP expression in the cells may alleviate ER stress in β cells.

In some embodiments, disclosed herein are methods for use in treating pancreatic cancer in a subject in need thereof, comprising inhibiting CHOP in pancreatic β cells by administering to the subject a composition comprising a CHOP inhibiting moiety and a pancreatic β cell targeting moiety (also may be interchangeably referred to as CHOP inhibiting moiety). In some embodiments, the inhibition of the endoplasmic reticulum (ER) stress transcription factor, CHOP, in pancreatic β cells, will normalize insulin secretion and improve glycemic control. In some embodiments, the inhibition of CHOP in β cell using a CHOP inhibiting moiety disclosed herein may lead to normalizing insulin secretion and improving glycemic control, may prevent pancreatic and liver steatosis.

In some embodiments, disclosed herein are various aspects of the disclosure that describe hyperinsulinemia driven endocrine-exocrine crosstalk, and which may generate primary PDAC and metastasis to the liver. In some embodiments, ER stress may also be connected to obesity and insulin resistance in T2D. In some embodiments, high-fat diet and obesity induce ER stress in the liver, which can suppress insulin signaling via c-Jun N-terminal kinase activation.

### Use of CHOP-Inhibiting Moiety in Kras Mutations

Kras is one of the most prolific oncogenes whose mutation has been associated with promoting and leading to the activation of or progression of cancers, especially in the colorectum, pancreas, lung, and blood plasma, with varying prevalence of specific activating missense mutations. In some embodiments, CHOP inhibiting moiety compositions, methods or treatments disclosed herein can prevent Kras-associated cancers. For example, inhibition of CHOP in β cell using CHOP inhibiting moiety can prevent pancreatic and liver steatosis in a subject carrying pancreas specific Kirsten rat sarcoma (Kras) mutations. T2DM may induce fibrosis in areas adjacent to islets, perturb and inflame islets and may promote excessive insulin signaling and tumor development in diabetic subjects with oncogenic Kras mutations. In some embodiments, expression of Kras mutation in pancreatic cells in cases of obesity may be tumor-promoting. In some embodiments, the inhibition of CHOP in β cell using CHOP inhibiting moiety disclosed herein, may lead to prevention of pancreatic and liver steatosis. Pancreatic and liver steatosis may both occur following consumption of a high-fat diet. In some embodiments, the inhibition of CHOP in β cell which may lead to prevention of pancreatic and liver steatosis, which may occur in cases where the subject has a condition of obesity. In some embodiments, obesity may promote hyperglycemia, and hyperinsulinemia, which may accelerate pancreatic intraepithelial neoplasia (PanIns) progression and PDAC tumor incidence.

In some embodiments, inhibition of CHOP in β cell using CHOP inhibiting moiety may normalize insulin secretion and glycemic control and may prevent pancreatic and liver steatosis in a subject that has consumed high-fat diets and in such a case, provision of a CHOP inhibiting moiety may reduce primary PDAC tumor growth and preventing liver metastasis.

### Aspects of a High-Fat HF Diet

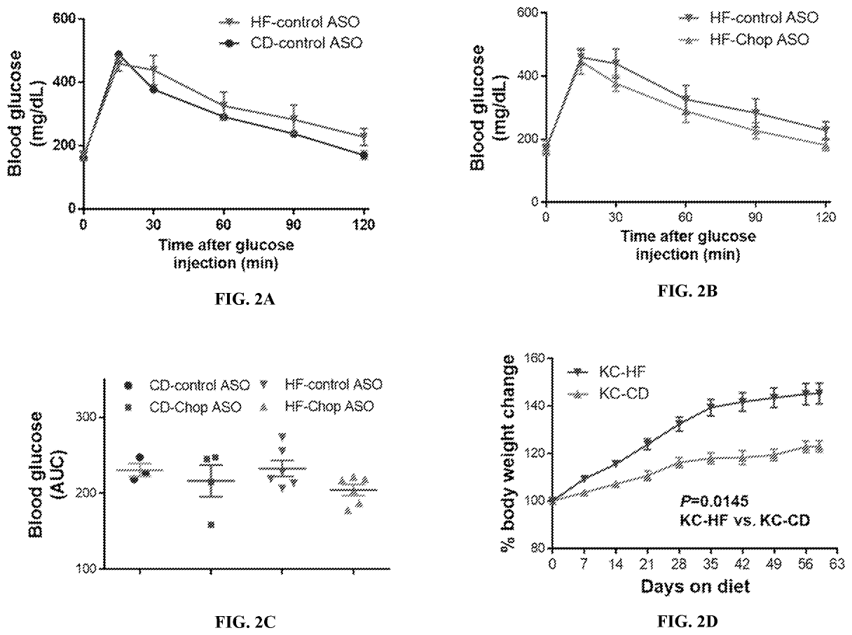

A high-fat diet promotes pancreatic and liver steatosis and growth of the primary and metastatic pancreatic tumors and impose body weight gain (FIG. 2). In some embodiments, these effects of high-fat diets may lead to marked accumulation of tumor-associated fibroblasts in the pancreas, extensive fibrosis and augmentation of the inflammatory or immune response.

A high-fat diet may impose fast progression of precancerous lesions (PanINs) and increase incidence of higher PDAC. In some embodiments, β cell dysfunction influences PDAC primary tumor development and pancreatic cancer metastasis to the liver. In some embodiments, provision of a CHOP inhibiting moiety can alleviate, reduce, slow, or abrogate PDAC primary tumor development. In some embodiments, the inhibition of CHOP in β cell by provision of a CHOP inhibiting moiety may reduce, lower, delay, or stop the initiation and progression of PDAC.

Because HF diets can lead to insulin dysregulation, high levels of local and circulating insulin (and insulin growth factor 1) can activate growth signaling and may promote survival and pro-fibroinflammatory responses in cancer and stromal cells (including fibroblast-type cells and immune cells), all which can be conducive to the development of primary tumors and metastases.

In some embodiments, the inhibition of CHOP using a CHOP inhibiting moiety disclosed herein may alleviate the many negative effects of consuming a high-fat diet (HF).

In some embodiments, HF can lead to the development of obesity, hyperglycemia, hyperinsulinemia and fatty liver. In some embodiments, fatty liver can lead to liver cirrhosis. HF diet may increase the rate of metastasis in PDAC cases and in some cases fatty liver may be more likely to support cancer cell growth and metastasis.

In some embodiments, HF may lead to development of liver steatosis and associated conditions. For example, HF diet can cause liver steatosis leading to marked transformation of the pancreas parenchyma which may be presented as landscape comprising neoplastic ducts, cancer cells, extensive stromal areas that are enriched in immune cells, fibroblasts. In some embodiments, administration of CHOP inhibiting moiety of the present disclosure following sustained HF diet for a period of time can prevent loss of normal parenchyma, reduce neoplastic duct numbers and can improve fatty liver. In some embodiments, provision of a CHOP inhibiting moiety may improve glucose tolerance even when the glucose level in a subject or mammal may initially be much more elevated due to consuming a HF diet for a period of time, for example, for at least one week, at least 2-3 weeks, or for one or more weeks, two or more weeks, more than 2-5 weeks, or more than 6 weeks, or for at least 7, 8, 9, 10, 20, 30, 40, 50, 60, 70, 80, or 90-1500 weeks or more than 1510 weeks.

Obesity can be associated with HF diet and may lead to alteration of the pancreas landscape and may expand the pancreatic intraepithelial neoplasia (PanIns) and stromal areas which may lead to a reduction in normal parenchyma. In some embodiments, administration of a CHOP inhibiting moiety in β cells of subjects consuming HF diets can lead to transformation of the pancreas parenchyma or may prevent the loss of normal parenchyma and reduce neoplastic duct numbers which can be seen in PanIN and PDAC progression. In some embodiments, HF diet can promote accumulation of myofibroblasts and type 1 macrophages mainly in stromal areas, while immunosuppressor type 2 macrophages may predominate in areas surrounding PanINs. As such, cytotoxic T lymphocytes can often be sparse following consumption of a HF diet.

Obesity, HF diets, insulin dysregulation or β cell dysfunction and the conditions and disorders or diseases associated with physiological dysregulation (for example, PDAC metastasis in the liver, pancreatic and liver steatosis, fatty liver disease, pancreatic and liver cancer) can lead to dramatic changes immunological, cellular and physiological functioning of the body. In some embodiments, physiological dysregulation can be observed at the molecular level following consumption of a HF diet such as for instance, dysregulation may contribute to differential expression of clusters of protein genes, which may be markers of β cell dysfunction or physiological perturbation. In some embodiments, obesity can be a risk factor for fibrosis of the pancreas and liver steatosis which can contribute to development of a fatty liver. In some embodiments, fatty liver may contribute to development of liver metastases.

In some embodiments, inhibition of CHOP in β cell may alleviate obesity-induced promotion of PDAC progression in a subject. In some embodiments, provision of a CHOP inhibiting moiety may lead to reduction of obesity-mediated conditions or disorders.

In some embodiments, CHOP may localize in the nucleus of neoplastic cells of human PDAC tumors. In some embodiments, CHOP in the neoplastic cells may be targeted by the compositions disclosed herein which may lead to inhibition of CHOP expression in the endoplasmic reticulum and which may subsequently alleviate ER stress associated with induction of the CHOP-induced transcription in β cells of the pancreas.

In some embodiments, an inhibitor of CHOP in β cells may reduce the acceleration of primary tumorigenesis in the pancreas. For example, a CHOP inhibiting moiety that inhibits CHOP in β cell in obese cases can improve fibrosis of the pancreas, improve, alleviate or reduce pancreatic and liver steatosis, and may reduce fatty liver. In some embodiments, inhibition of CHOP in β cell may delay, reduce, or abrogate macro and micro steatosis in the pancreas or liver or both.

In some embodiments, provision of a CHOP inhibiting moiety may alleviate or improve HF-diet induced and obesity-associated immunosuppressive microenvironment. In some embodiments, provision of a CHOP inhibiting moiety may promote accumulation of myofibroblasts and type 1 macrophages mainly in the stroma areas, while suppressing type 2 macrophage predominant in the areas surrounding PanIN. In some embodiments, provision of CHOP inhibiting moiety disclosed herein may result in induction and availability of immune effector T cells, for instance, induction of T cells, CD8+ T cells, or cytotoxic T lymphocytes, natural killer cells, or any other immune cells where induction leads to improvement in the immunological, cellular, physiological, molecular response, psychological, mental, emotional well-being in general. In some embodiments, provision of CHOP inhibiting moiety disclosed herein can result in improved response in a tumor microenvironment, for instance, immunological response that may reduce the areas enriched with PanIN and improve, reduce or abrogate PDAC metastasis to the liver, or may alleviate or reduce pancreatic and liver steatosis or cancer in general.

In some embodiments, silencing or inhibiting CHOP in β cell using CHOP inhibitors disclosed herein may provide a target for alleviating dysregulated insulin secretion and fatty liver disease in cases of diabetes.

In some embodiments, inhibition of CHOP in pancreatic β cells using a CHOPs inhibiting moiety disclosed herein may normalize insulin secretion. In some embodiments, inhibition of CHOP in pancreatic β cells may lead to glycemic control. In some embodiments, inhibition of CHOP in pancreatic β cells may prevent pancreatic and liver steatosis subjects consume HF diet or not, or when subjects harbor the pancreas specific Kras mutation or not. In some embodiments, inhibition of CHOP in pancreatic β cells may lead to reduction of primary PDAC tumor growth and prevent liver metastasis. In some embodiments, inhibiting CHOPs in pancreatic β cells may slow growth of PDAC tumors and prevent recurrence of metastasis. In some embodiments a subject population with PDAC may also have disorders of glucose homeostasis and may benefit from the therapeutic compositions described herein. In some embodiments, disorders of glucose homeostasis can be unique among human cancers. Although current therapies can affect glucose homeostasis, they are not aimed at fatty liver or fatty pancreas which may promote PDAC and its metastasis. In some embodiments, that inhibition or silencing of CHOP can lead to therapeutic benefits for the pancreatic β cell which can slow primary PDAC tumor growth and prevent metastasis.

With this disclosure, it is to be understood that the methods of use, compositions, kits, formulations, pharmaceuticals, products and other aspects of the present disclosure are not limited to particular exemplary embodiments described herein, and as such, may of course, vary.

### Dosage and Administration of a CHOP-Inhibiting Moiety

In some embodiments, a high-fat diet comprising 45% fat may facilitate metastasis in the liver of a subject or mammal harboring Kras mutations, which can lead to rapid development of obesity, development of mild hyperglycemia, hyperinsulinemia and fatty liver. In some embodiments, administration of a CHOP inhibiting moiety following a period of feeding on a high-fat diet may lead to improved glucose metabolism and improvement in hyperglycemia. In some embodiments a high-fat diet may comprise less than 45% fat. In some embodiments, a high-fat diet may comprise more than 45% fat. In some embodiments, a high-fat diet may comprise about 45% fat. In some embodiments, a high-fat diet may comprise between 10% to 80% fat. In some embodiments, a high-fat diet may comprise least than 30% fat. In some embodiments, a high-fat diet may comprise at least 30% fat. In some embodiments, a high-fat diet may comprise over 80% fat.

In some embodiment, the period of feeding on a high diet may be for less than 1 week, more than 1 week, or may be 1-5 weeks, or 2-5 weeks, or 3-5 weeks, or over 5 weeks, 5-10 weeks, or greater than 10 weeks, prior to administration of a CHOP inhibiting moiety. In some embodiments, the administration of a CHOP inhibiting moiety following a period on a high-fat diet may comprise administration of a CHOP inhibiting moiety at a dose of 0.1 to 5 mg/Kg body weight for human subject given once, twice or more times per day, per week and given for as long as required, which could mean anywhere from the day of administrating the regimen to a day or more later, a week, or the lifetime of the subject. In some embodiments, the administration of a CHOP inhibiting moiety following a period on a high-fat diet may comprise administration of a CHOP inhibiting moiety at a dose of less than 0.1 mg/Kg body weight for human subject given once, twice or more times per day, per week and given for as long as required, which could mean anywhere from the day of administrating the regimen to a day or more later, a week, or the lifetime of the subject.

In some embodiments, the administration of a CHOP inhibiting moiety following a period on a high-fat diet may comprise administration of a CHOP inhibiting moiety at a dose of greater than 5 mg/Kg body weight for human subject, given once, twice or more times per day, per week and given for as long as required, which could mean anywhere from the day of administrating the regimen to a day or more later, a week, or the lifetime of the subject. In some embodiments, a dose of a CHOP inhibiting moiety can be administered for a period of 4 weeks following the high-fat diet or any time before a high-fat diet. In some embodiments, a dose of a CHOP inhibiting moiety can be administered for a period of less than one week, or 1-5 weeks, at least one week, or 2, 3, 4, 5, 6, 7, 8, 9, 10 weeks, or more than 10 weeks, or 4 weeks to 52 weeks, or more than 1-5 years or more than 5 years following the high-fat diet or before a high-fat diet. Administration of a CHOP inhibiting moiety may be provided via various routes. In some embodiments, a CHOP inhibiting moiety may be administered via parenteral route, intramuscular injection or via any other means of administrating as disclosed elsewhere in the present disclosure (For a review of the dosing regimen, please refer to the below reference: Mohammad Shadid, Mohamed Badawi & Abedelnasser Abulrob (2021): Antisense oligonucleotides: absorption, distribution, metabolism, and excretion, Expert Opinion on Drug Metabolism & Toxicology, DOI: 10.1080/17425255.2021.1992382.

In some embodiments, administration of a CHOP inhibiting moiety can alleviate or improves glucose tolerance. In some embodiments, administration of a CHOP inhibiting moiety disclosed herein may lower body weight. In some embodiments, said administration of a CHOP inhibiting moiety that may lower body weight, may do so within at least 1 day, 2 days, 3 days, 4 days, 5 days, 6 days, 7 days, within about 1-2 weeks, about 2-5 weeks, about 5-10 weeks, more than 10 weeks following administration of a CHOP inhibiting moiety. In some embodiments, administration of a CHOP inhibiting moiety lowers body weight in at least 10 weeks to within 1-2 years, or more than 2 years after administration of the CHOP inhibiting moiety. In some embodiments, administration of a CHOP inhibiting moiety may alleviate or improve glucose tolerance. In some embodiments, improvement of glucose tolerance can occur within at least 1 day or 2, 3, 4, 5, 6, 7 days after administration of a CHOP inhibiting moiety in β cell. In some embodiments, improvements that may lead to glucose control can occur within at least 1 week, or 1-2 weeks, 3-5 weeks, or within at least 2-10 weeks, or more than 10 weeks, 20 weeks, 30 weeks, 40 weeks, 50 weeks, or more 60 weeks after administration of a CHOP inhibiting moiety disclosed herein.

### Definitions

As such it is to be understood that the terminology used herein is for the purpose of describing particular cases only and is not intended to be limiting. In this application, the use of the singular includes the plural unless specifically stated otherwise. As used herein, the singular forms “a,” “an,” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

The term “about” or “approximately” may mean within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the present disclosure, and vice versa. Furthermore, compositions of the present disclosure may be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Reference in the specification to a “cell” may refer to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.gf., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell may be a synthetically made, sometimes termed an artificial cell).

Reference in the specification to “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled by well-known techniques. Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,NcN′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides may include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAN1RA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, TR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-1 4-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides may also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-cICTP, biotin-14-dCTP), and biotin-dUTP (e.g., biotin-11-dUTP, biotin-1.6-dUTP, biotin-20-dUTP).

Terms such as “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, eDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

Reference in the specification to “conjugated” may be used to designated chemically bonded i.e., attached by chemical bonds. A conjugate is a molecule, example a peptide that is chemically (for example covalently) linked to a biomolecule or molecule of interest, for example, a nucleic acid, that is conjugated to another molecule.

Reference in the specification to “operably linked” refers to a functional relationship between two or more nucleic acid segments. Typically, it refers to the functional relationship of a transcriptional regulatory sequence to a transcribed sequence.

“Polyadenylation sequence” (also referred to as a “poly A⁺ site” or “poly A⁺ sequence”) refers to a DNA sequence which directs both the termination and polyadenylation of the nascent RNA transcript. Efficient polyadenylation of the recombinant transcript is desirable, as transcripts lacking a poly A⁺ tail is typically unstable and rapidly degraded. The poly A⁺ signal utilized in an expression vector may be “heterologous” or “endogenous”. An endogenous poly A⁺ signal is one that is found naturally at the 3′ end of the coding region of a given gene in the genome. A heterologous poly A⁺ signal is one which is isolated from one gene and placed 3′ of another gene, e.g., coding sequence for a protein. A commonly used heterologous poly A⁺ signal is the SV40 poly A⁺ signal. The SV40 poly A⁺ signal is contained on a 237 bp BamHI/BclI restriction fragment and directs both termination and polyadenylation; numerous vectors contain the SV40 poly A⁺ signal. Another commonly used heterologous poly A⁺ signal is derived from the bovine growth hormone (BGH) gene; the BGH poly A⁺ signal is also available on a number of commercially available vectors. The poly A⁺ signal from the Herpes simplex virus thymidine kinase (HSV tk) gene is also used as a poly A⁺ signal on a number of commercial expression vectors. The polyadenylation signal facilitates the transportation of the RNA from within the cell nucleus into the cytosol as well as increases cellular half-life of such an RNA. The polyadenylation signal is present at the 3′-end of an mRNA.

Reference in the specification to “exon” refers to a nucleic acid sequence found in genomic DNA that is bioinformatically predicted and/or experimentally confirmed to contribute contiguous sequence to a mature mRNA transcript.

Reference in the specification to “intron” refers to a sequence present in genomic DNA that is bioinformatically predicted and/or experimentally confirmed to not encode part of or all of an expressed protein, and which, in endogenous conditions, is transcribed into RNA (e.g., pre-mRNA) molecules, but which is spliced out of the endogenous RNA (e.g., the pre-mRNA) before the RNA is translated into a protein.

Reference in the specification to “complement,” “complements,” “complementary,” and “complementarity,” as used herein, can refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/embossneedle/nucleotide.html), the BLAST algorithm (see e.g., the BLAST alignment tool available at blast.ncbi.nlm.nih.gov/Blast.cgi, optionally with default settings), or the Smith-Waterman algorithm (see e.g., the EMBOSS Water aligner available at www.ebi.ac.ukaools/psa/emboss_water/nucleotide.html, optionally with default settings). Optimal alignment can be assessed using any suitable parameters of a chosen algorithm, including default parameters. Complementarity may be perfect or substantial/sufficient. Perfect complementarity between two nucleic acids may mean that the two nucleic acids may form a duplex in which every base in the duplex is bonded to a complementary base by Watson-Crick pairing. Substantial or sufficient complementary may mean that, a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions may be predicted by using the sequences and standard mathematical calculations to predict the melting temperature (Tₘ) of hybridized strands, or by empirical determination of Tₘ by using routine methods.

The term “knockout” (“KO”) or “knocking out” as used herein refers to a deletion, deactivation, or ablation of a gene in a cell, or in an organism, such as, in a pig or other animal or any cells in the pig or other animal. KO, as used herein, may also refer to a method of performing, or having performed, a deletion, deactivation or ablation of a gene or portion thereof, such that the protein encoded by the gene is no longer formed.

The term “knockin” (“KI”) or “knocking in” as used herein refers to an addition, replacement, or mutation of nucleotide(s) of a gene in a pig or other animal or any cells in the pig or other animal. KI, as used herein, may also refer to a method of performing, or having performed, an addition, replacement, or mutation of nucleotide(s) of a gene or portion thereof.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Reference in the specification to “derivative,” “variant,” and “fragment,” may be with regards to a polypeptide, can indicate a polypeptide related to a wild-type polypeptide, for example either by amino acid sequence, structure (e.g., secondary and/or tertiary), activity (e.g., enzymatic activity) and/or function. Derivatives, variants and fragments of a polypeptide may comprise one or more amino acid variations (e.g., mutations, insertions, and deletions), truncations, modifications, or combinations thereof compared to a wild-type polypeptide.

Reference in the specification to “percent (%) identity,” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps may be introduced in one or both of the candidate and reference sequences for optimal alignment and non-homologous sequences may be disregarded for comparison purposes). Alignment, for purposes of determining percent identity, may be achieved in various ways that are within the skill in the art, for instance, using publicly available computer software such as BLAST, ALIGN, or Megalign (DNASTAR) software. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

Reference in the specification to “nucleic acid editing moiety,” can indicate a moiety, which may induce one or more gene edits in a polynucleotide sequence. The polynucleotide sequence may be in a host cell. Alternatively, the polynucleotide sequence may not be in a host cell. Gene editing using the nucleic acid editing moiety may comprise introducing one or more heterologous polynucleic acids (for example, genes, or fragments thereof) in a cell, or deleting one or more endogenous polynucleic acids (for example, genes, or fragments thereof) from the cell. In some cases, gene editing using the nucleic acid editing moiety may comprise substituting any one or more polynucleic acids (for example, genes, or fragments thereof) thereof. In some cases, gene editing using the nucleic acid editing moiety may comprise a combination of any of the above, either simultaneously or sequentially. In some cases, the one or more polynucleic acids may be a DNA. In some cases, the one or more polynucleic acids may be genomic DNA. In some cases, the any one or more genes or nucleic acid portions thereof may be added to or deleted from the chromosomal DNA of a cell by the nucleic acid editing moiety. In some cases, the one or more polynucleic acids may be genomic DNA. In some cases, one or more polynucleic acids may be added to or deleted from the chromosomal DNA of a cell by the nucleic acid editing moiety, that is not part of a gene. In some cases, the one or more polynucleic acids may be contained in exosomes. In some cases, one or more polynucleic acids may be in mitochondria or any other cell organelle. In some cases, the any one or more genes or nucleic acid portions thereof may be added to or deleted from the episomal DNA or epichromosomal DNA of the cell by the nucleic acid editing moiety. In some cases, one or more polynucleic acids may be RNA. In some cases, one or more exogenous polynucleic acids may be added into the genomic DNA, via integration of the exogenous polynucleic acids into the genomic DNA. Integration of any one or more genes into the genome of a cell may be done using any suitable method. Non-limiting examples of suitable methods for the genomic integration and/or genomic replacement strategies disclosed and described herein include CRISPR-mediated genetic modification using Cas9, Cas12a (Cpf1), or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof. Designer zinc fingers, transcription activator-like effectors (TALEs), or homing meganucleases are available for producing targeted genome perturbations.

Targeted genome editing is possible via CRISPR-mediated genetic modification using a Cas or Cas-like endonuclease. CRISPR (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli, and associated genes. Similar interspersed SSRs may be identified in Haloferax mediterranei, Streptococcus pyogenes, Anabaena, and Mycobacterium tuberculosis. The CRISPR loci typically differ from other SSRs by the structure of the repeats, which have been termed short regularly spaced repeats (SRSRs). The repeats are short elements that occur in clusters that are regularly spaced by unique intervening sequences with a substantially constant length. Although the repeat sequences are highly conserved between strains, the number of interspersed repeats and the sequences of the spacer regions typically differ from strain to strain. CRISPR loci have been identified in more than 40 prokaryotes including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

Cas9 gene may be found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region. A Cas 9 protein may be from an organism from a genus comprising, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, or Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus.

The nucleic acid editing moiety may comprise a nucleic acid cleavage moiety. The nucleic acid cleavage moiety may introduce a break or a cleavage in a nucleic acid site molecule. The nucleic acid cleavage moiety may be capable of recognizing a specific cleavage recognition site, for example, when in proximity to the cleavage recognition site on a target polynucleotide sequence. In some cases, the nucleic acid cleavage moiety may be directed by a second molecule (such as a nucleic acid, e.g., sequence specific guide RNA for Cas9) to a specific cleavage site on a polynucleic acid, for introducing a break or cleavage on the polynucleic acid. The nucleic acid cleavage moiety may initiate an introduction, deletion or substitution of the nucleic acid in the genomic DNA. In some cases, the nucleic acid cleavage moiety is a nuclease, or a functional fragment thereof. In some cases, the nucleic acid cleavage moiety may comprise an endonuclease, an exonuclease, a DNase, an RNase, a strand-specific nuclease, or a more specialized nuclease, (for example, a CRISPR associated protein 9, Cas 9), or any fragment thereof. In some cases, the nucleic acid cleavage moiety may be nickase.

In some cases, the nuclease is an AAV Rep protein, Rep68/78.

In some cases, a nucleic acid editing moiety may comprise a viral machinery or a fragment thereof that is capable of incorporating a viral gene into a host cell. For example, a nucleic acid editing moiety may refer to a viral integrase system, such as a lentiviral integrase system. Integrase is a retroviral enzyme that catalyzes integration of DNA into the genome of a mammalian cell, a useful step of retrovirus replication in the retroviral infection process. The process of integration can be divided into two sequential reactions. The first one, named 3′-processing, corresponds to a specific endonucleolytic reaction which prepares the viral DNA extremities to be competent for the subsequent covalent insertion, named strand transfer, into the host cell genome by a trans-esterification reaction. In some cases, a nucleic acid editing moiety may additionally refer to a transposon/transposase or a retrotransposase system or a component thereof, for integration of a piece of DNA into the genome. However, inserting exogenous DNA into specific genomic sequences is preferred over random and semi-random integration throughout the target cell's genome, such as with some retroviral vectors and transposons/transposases. The random and semi-random integration procedures may result in outcomes such as positional-effect variegation, transgene silencing, and, in some cases, insertional mutagenesis caused by transcriptional deregulation or physical disruption of endogenous target-cell genes.

Reference in the specification to antisense oligomeric nucleic acids or antisense oligonucleotides or ASOs refers to antisense RNA, that can be synthetic single-stranded deoxyribonucleotide analogs, usually 15-30 bp in length. In some embodiments, the antisense RNA is 10-50 nucleotides in length. In some embodiments, the antisense RNA is 15-45 nucleotides in length. In some embodiments, the antisense RNA is 20-50 nucleotides in length. In some embodiments, the antisense RNA is 20-40 nucleotides in length. In some embodiments, the antisense RNA is 15-40 nucleotides in length. In some embodiments, the antisense RNA is 10-30 nucleotides in length. In some embodiments, the antisense RNA is 20-30 nucleotides in length. In some embodiments, the antisense RNA is 22-30 nucleotides in length. In some embodiments, the antisense RNA is 20-27 nucleotides in length. In some embodiments, the antisense RNA is 21-27 nucleotides in length. In some embodiments, the oligomeric nucleic acids is single stranded. In some embodiments, the oligomeric nucleic acids sequence (3′ to 5′) is antisense and complementary to the sense sequence of the target nucleotide sequence. In some embodiments, the oligomeric nucleic acids sequence is a double stranded ribonucleic acid, e.g., an siRNA, also called an iRNA. In some embodiments, the antisense oligomer comprises modified and/or unmodified nucleotides. In some embodiments, unmodified oligonucleotides after quick degradation by circulating nucleases are excreted by the kidney; unmodified oligonucleotides are generally too unstable for therapeutic use. Therefore, chemical modification strategies have been developed to overcome this and other obstacles in ASO therapy program. Commonly used modification in these ASOs is 2′ ribose modifications that include 2′-O-methoxy (OMe), 2′-O-methoxy-ethyl (MOE), and locked nucleic acid (LNA). 2′-OMe modifications are commonly used in a ‘gapmer’ design, which is a chimeric oligo comprising a DNA sequence core with flanking 2′-MOE nucleotides that enhances the nuclease resistance, in addition to lowering toxicity and increasing hybridization affinities. Sequence specific “small inhibiting RNA (siRNA)” or “iRNA” relates to small RNA sequences that bind to a target nucleic acid molecule, which can expression of a gene expression product. Introduction of double-stranded RNA (dsRNA) also called interfering RNA (RNAi), or hairpin RNA is an effective trigger for the induction of gene silencing in a large number of eukaryotic organisms, including animals, fungi, and plants. Both the qualitative level of dsRNA-mediated gene silencing (i.e., the level of gene silencing within an organism) and the quantitative level (i.e., the number of organisms showing a significant level of gene silencing within a population) have proven superior to the more conventional antisense RNA or sense RNA mediated gene silencing methods. In some embodiments, the antisense oligonucleotide (ASO) comprises at least 5 consecutive nucleotides that are complementary to and antisense of a nucleic acid sequence encoding a human protein, such as a C/EBP homologous protein. In some embodiments, the antisense oligonucleotide (ASO) comprises at least 6, 7, 8, 9, 10, 11, 12, 13, 14, 15, 16, 17, 18, 19, 20, 21, 22, 23, 24, 25, 26, 27, 28, 29, or at least 30 consecutive nucleotides that are complementary and antisense of a nucleic acid sequence encoding a human C/EBP homologous protein. In some embodiments, the ASO is an siRNA, having 21-27 nucleotide pairs, and at least one nucleotide overhang at the 5′ and the 3′ end.

In some embodiments the ASO is a single stranded oligomer. In some embodiments the single stranded oligomer comprises 20-50 nucleotides in length. In some embodiments the single stranded oligomer comprises 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 nucleotides.

Another method of inhibiting gene expression comprises targeting a nucleic acid molecule to an anti-sense transcript and sense strand transcript, wherein the nucleic acid molecule targeting the anti-sense transcript is complementary to the anti-sense strand and the nucleic acid molecule targeting the sense transcript is complementary to the sense strand, and binding of the nucleic acid to the anti-sense and sense transcript, thereby, inhibits gene expression. The nucleic acid molecule is an RNA molecule and the nucleic acid molecules targeting the anti-sense and sense transcripts bind said transcripts in convergent, divergent orientations with respect to each other and/or are overlapping. Method for gene suppression in eukaryotes by transformation with a recombinant construct containing an antisense and/or sense nucleotide sequence for the gene(s) to be suppressed is known in the art.

The present disclosure provides methods and compositions for targeting CHOP expression, which may be associated with insulin secretion in ER stress, downstream of unfolded protein response.

In one aspect pancreatic β cell CHOP may be targeted. In the aspect, provided herein is a method of inhibiting C/EBP homologous protein (CHOP) in pancreatic β cells by administering to the subject a composition comprising: (a) a CHOP inhibiting moiety, and (b) a pancreatic β cell targeting moiety. A pancreatic β cell CHOP may be inhibited by targeted deletion of pancreatic β cell CHOP. This may be achieved by any method, including but not limiting to using site specific nucleases and recombinases to direct and delete CHOP in pancreatic beta cells using transposons, retrotransposons, TALENs, zinc finger proteins, CRISPR-Cas systems or Cre-lox systems or viral integrase systems.

In some embodiments, the CHOP inhibiting moiety is a nucleic acid. In some embodiments, the CHOP inhibiting moiety is a DNA. In some embodiments, the CHOP inhibiting moiety is an RNA. In some embodiments, the CHOP inhibiting moiety is an inhibitory RNA. In some embodiments, the RNA is an antisense oligomeric nucleic acid, also called antisense oligonucleotide or ASO. In some embodiments, the RNA is a double stranded molecule capable of inhibiting CHOP expression. In some embodiments the RNA is a single stranded structure capable of inhibiting CHOP expression. In some embodiments, the RNA is an antisense oligomeric nucleic acid capable of reducing or inhibiting the expression of pancreatic β cell CHOP mRNA. In some embodiments the ASO is about 10-1000 nucleotides long. In some embodiments the nucleic acid is 10-500 nucleotides long, 10-400 nucleotides long, 10-300 nucleotides long, 10-200 nucleotides long, or 10-100 nucleotides long or any length in between. In some embodiments the ASO is about 20, 25, 30, 35, 40, 45, 50, 55, 60, 65, 70, 75, 80, 85, 90, 95, 100, 105, 110, 120, 130, 140, 150, 160, 170, 180, 190, 200, 205, 210, 215, 220, 225, 230, 235, 240, 245, 250, 255, 260, 265, 270, 275, 280, 285, 290, 295 or about 300 nucleotides long. In some embodiments the ASO is less than 100 nucleotides long, less than 90, or less than 80, or less than 70, or less than 60, or less than 50, or less than 40, or less than 30 nucleotides long or any length in between. In some embodiments the nucleic acid is 10-50 nucleotides long, or 12-45, or 15-30 nucleotides long.

In some embodiments, the CHOP inhibiting moiety comprises a nucleic acid sequence that has about at least 80% sequence identity to a contiguous stretch of nucleotides of the length of the ASO within an mRNA encoding CHOP. In some embodiments, the ASO comprises a nucleic acid sequence that that has about at least 80% sequence identity to a contiguous stretch of nucleotides of the length of the ASO within an mRNA encoding a human CHOP. In some embodiments, the inhibitory RNA has a sequence homology or complementarity with at least 10, at least 12, at least 14, at least 16, at least 18, at least 20, at least 25, at least 30, at least 35, at least 40, at least 45 or at least 50 contiguous nucleotides of the CHOP gene sequence or the CHOP mRNA.

In some embodiments, the CHOP inhibiting moiety is a single stranded RNA.

In some embodiments, the CHOP inhibiting moiety is a morpholino antisense oligomer.

In some embodiments, the CHOP inhibiting moiety is a double stranded RNA.

In some embodiments, the CHOP inhibiting moiety comprises an RNA that comprises at least one or more modified bases. In some embodiments, the modified base is a pseudouridine, or 2-methyl cytosine. In some embodiments, the RNA may be stabilized, by one or more of i) substituting at least one naturally occurring nucleotide base with a modified base, ii) conjugating with a biomolecule, such as a peptide.

In some embodiments the pancreatic β cell targeting moiety is a protein, peptide or nucleic acid that can direct the CHOP inhibiting moiety to the pancreatic cell. In some embodiments the pancreatic β cell targeting moiety is an antibody that can be conjugated to the chop inhibiting moiety. In some embodiments the pancreatic β cell targeting moiety is a peptide. The peptide may be conjugated to the antisense oligomer. In some embodiments, the peptide is conjugated to a linker which links the peptide at one end and the oligonucleotide in the other. The linker may be a chemical crosslinker. There are several synthetic or chemical cross-linkers.

In some embodiments, the peptide stabilizes the ASO. In some embodiments, the nucleic acid is targeted to a particular cell by the peptide. In some embodiments, the targeting moiety is a molecule capable of binding to a receptor on pancreatic β cell and be internalized into the cell. In some embodiments, the peptide is a GLP-1 peptide, or a fragment thereof. In some embodiments, the peptide comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids.

Alternatively, in some embodiments, the CHOP inhibiting moiety is a DNA sequence. The DNA sequence may comprise a modified nucleotide. The DNA sequence may encode one or more protein of interest, which when expressed, can inhibit the expression of CHOP in a pancreatic β cell. In some embodiments, the DNA may comprise a sequence encoding a nuclease, an integrase, or a recombinase.

Alternatively, the DNA may comprise a sequence, which can be inserted into a certain locus of a target cell by a targeting moiety. The targeting moiety in this case may be a nuclease, an integrase, or a recombinase. The DNA may comprise a nucleic acid sequence, which may be introduced within a gene or a chromosomal locus by the action of the targeting moiety, can inhibit the expression of CHOP in a pancreatic β cell.

In one aspect, the targeting of the pancreatic β cell CHOP may be regulated. Provided herein is a method of regulating pancreatic β cell CHOP expression, comprising administering a nucleic acid composition comprising a CHOP inhibitor moiety and a pancreatic β cell targeting moiety. The regulating can be achieved by regulating the induction of CHOP inhibitor moiety and a pancreatic β cell targeting moiety. In one embodiment, the induction of an ASO may be regulated by designing an ASO expression construct that comprises a promoter operably linked to the ASO, wherein the promoter is inducible by a regulator. In some embodiments the targeting moiety may be designed such that the expression of the targeting moiety is dependent on activation by a regulator. A large number of vector and promoter systems are well known in the art. Construction of expression vectors having a promoter that is inducible by a regulator is known to one of skill in the art. Exemplary inducible promoter may be a doxycycline or a tetracycline inducible promoter. Tetracycline regulated promoters may be both tetracycline inducible or tetracycline repressible, called the tet-on and tet-off systems. The tet regulated systems rely on two components, i.e., a tetracycline-controlled regulator (also referred to as transactivator) (tTA or rtTA) and a tTA/rtTA-dependent promoter that controls expression of a downstream cDNA, in a tetracycline-dependent manner. tTA is a fusion protein containing the repressor of the Tn10 tetracycline-resistance operon of Escherichia coli and a carboxyl-terminal portion of protein 16 of herpes simplex virus (VP16). The tTA-dependent promoter consists of a minimal RNA polymerase II promoter fused to tet operator (tetO) sequences (an array of seven cognate operator sequences). This fusion converts the tet repressor into a strong transcriptional activator in eukaryotic cells. In the absence of tetracycline or its derivatives (such as doxycycline), tTA binds to the tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. In contrast, in the tet-ON system, a mutant form of tTA, termed rtTA, has been isolated using random mutagenesis. In contrast to tTA, rtTA is not functional in the absence of doxycycline but requires the presence of the ligand for transactivation. A Tamoxifen inducible system may comprise a reversible switch, that can provide reversible control over the transcription of a gene or genes that are regulated by the system. The tamoxifen/estrogen receptor regulatable system can allow spatiotemporal control of gene expression, especially when combined with the Cre/Lox recombinase system, where the Cre recombinase is fused to a mutant form of the ligand-binding domain of the human estrogen receptor resulting in a tamoxifen-dependent Cre recombinase.

In one aspect, provided herein are compositions comprising a nucleic acid sequence capable for suppressing human CHOP gene expression, operably linked to a peptide. In some embodiments, the composition further comprises a targeting moiety that directs the nucleic acid sequence that inhibits CHOP expression to a target in a pancreatic cell.

The nucleic acid sequence capable for suppressing or inhibiting human CHOP gene expression in vivo is described in the previous paragraphs. In some embodiments, the nucleic acid sequence capable for suppressing human CHOP gene expression is an RNA. In some embodiments, the nucleic acid sequence capable for suppressing human CHOP gene expression is an inhibitory RNA. In some embodiments, the nucleic acid sequence capable for suppressing human CHOP gene expression is an antisense oligomeric RNA. In some embodiments, the nucleic acid sequence capable for suppressing human CHOP gene expression is an iRNA. In some embodiments the nucleic acid sequence capable for suppressing or inhibiting human CHOP gene expression is a double stranded RNA comprising of about 22 to about 28 nucleotides, and comprises at least one overhang, wherein at least the overhang is at the 5′ end or the 3′ end.

In some embodiments, the compositions provided herein are for use in selectively inhibiting CHOP in a pancreatic β cell. In some embodiments, one or more nucleic acid sequences may be incorporated in a vector. In some embodiments, the vector for expression of the recombinant protein is of a viral origin, namely a lentiviral vector or an adenoviral vector. In some embodiments, the nucleic acid encoding the recombinant nucleic acid is encoded by a lentiviral vector. In some embodiments the viral vector is an Adeno-Associated Virus (AAV) vector.

Alternatively, in one embodiment, the nucleic acid composition may be delivered inside a cell via a lipid vehicle, such as a liposome or a lipid nanoparticle. Lipid nanoparticles (LNP) may comprise a polar and or a nonpolar lipid. In some embodiments, cholesterol is present in the LNPs for efficient delivery. LNPs are 100-300 nm in diameter provide efficient means of mRNA delivery to various cell types; or can be administered. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiments, the LNP encapsulates the nucleic acid wherein the nucleic acid is inserted in a vector, such as a plasmid vector. In some embodiments, the LNP is used to deliver the nucleic acid into a subject. LNP can be used to deliver nucleic acid systemically in a subject. It can be delivered by injection. In some embodiments, the LNP comprising the nucleic acid is injected by intravenous route. In some embodiments the LNP is injected subcutaneously.

In one embodiment, provided herein is a method for treating pancreatic cancer, the method comprising inhibiting C/EBP homologous protein (CHOP) in pancreatic β cells by administering to the subject a composition comprising: (a) a CHOP inhibiting moiety, and (b) a pancreatic β cell targeting moiety. The composition, as described in the previous paragraphs, comprising a CHOP inhibiting moiety and a pancreatic β cell targeting moiety can be administered to the subject systemically. Administering the composition may be associated with the reduction of one or more conditions associated with T2D such as PDAC.

In one embodiment, administering the composition may be associated with reduction of pancreatic ER stress. In some embodiments, administering the composition may be associated with reduction in Ins1 and Ins2 gene expression, without disrupting normal pancreatic β cell function, or identity or normal gene expression. In some embodiments, the administration may be associated with at least 30% reduction in the expression of Ins1 and/or Ins2 gene expression. In some embodiments, the administration may be associated with at least 35%, 40%, 45%, 50% or 60% reduction in the expression of Ins1 and/or Ins2 gene expression. In some embodiments, the method is associated with reduction of UPR related gene expression in the pancreas. In some embodiments, the method is associated with reduction of UPR related gene expression in the liver. In some embodiments, the method is associated with reduction of UPR related gene expression in the pancreas and liver.

Provided herein is a method of treating pancreatic cancer in a subject in need thereof, comprising: inhibiting C/EBP homologous protein (CHOP) in pancreatic β cells by administering to the subject a composition comprising: (a) a CHOP inhibiting moiety, and (b) a pancreatic β cell targeting moiety. In some embodiments, the subject is a mammal. In some embodiments, the subject is a human. In some embodiments, the subject is a non-human mammal. In some embodiments, inhibiting CHOP in pancreatic β cells by administering to the subject the composition as described in the previous paragraphs may be associated with reduction in hepatomegaly.

Provided herein is a pharmaceutical composition comprising the composition as described above and a pharmaceutically acceptable excipient. Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). Acceptable carriers are physiologically acceptable to the administered patient and retain the therapeutic properties of the compounds with/in which it is administered. Acceptable carriers and their formulations are generally described in, for example, Remington' pharmaceutical Sciences (18^{th} ed. A. Gennaro, Mack Publishing Co., Easton, PA 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes. In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage. The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution there.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1. Determination of β Cell Dysfunction Influences on PDAC Primary Tumor Development

In this example, investigations were carried out to determine whether pancreatic β cell dysfunction influences PDAC primary tumor development. This particular aim was conducted over a series of experiments. First, the effect of CCAAT/enhancer-binding protein (C/EBP) homologous protein (CHOP) antisense oligomer (CHOP ASO) treatment on glucose metabolism was conducted. To do this, the metabolic effect of feeding a GLP1-conjugated CHOP AntiSense Oligonucleotides (GLP1-CHOP-ASO) on the metabolism of glucose was explored in KC mice, a model for obesity-induced promotion of PDAC progression, (KC mice are genetically engineered to carry the Kras mutation (K-ras is a key proto-oncogene that is usually altered in 90% of PDAC) and the bacterial recombinase Cre resulting in the KC with the P48+/Cre;Kras+/G12D mutations. (The KC mouse model has been described in the following references: Boj S F, Hwang C-I, Baker L A, et al. Organoid models of human and mouse ductal pancreatic cancer. Cell 2015; 160(1e2):324e38; Hingorani et al, 2003; Yong J et al 2021; Peng Y et al, 2020. The references Yong J. et al, 2021 and Peng Y et al., 2021) by feeding the KC mice a high-fat diet (HF; 45% fat) or a control diet (CD) for a period of 3 months starting shortly after weaning. After 2 months on the diets, a subset of mice received GLP1-CHOP-ASO intravenously (IV) given once per week for 4 weeks. Provision of the GLP1-CHOP-ASO to a subset of mice following 2 months on the above diets was conducted in order to determine the effects of β cell ER stress and increased insulin secretion as drivers promoting PDAC development, as well as to assess the effect of CHOP gene silencing in β cells. During the feeding exercise, mice were maintained on either a HF diet or CD during the time of administering a GLP1-CHOP-ASO treatment.

Following the above procedure, at the end of 4 weeks on GLP1-CHOP-ASO treatment, mice are sacrificed in order to: (1) assess pancreatic steatosis, PanIN progression, cancer incidence, stromal expansion and immune cell profiles using histological analysis, and assess; (2) measure β cell function, UPR activation in β cells and the exocrine compartment, glucose-stimulated insulin secretion, pancreatic and circulating insulin levels and other measurements to assess glycemic control; (3) evaluate islet mass, size and cellular organization to determine the potential reciprocal impact of HF and PDAC progression on the endocrine compartment; (4) analyze liver histology to assess hepatic steatosis triglyceride content, and the presence of metastasis.

### Results

By providing mice with the GLP1-conjugated CHOP Antisense Oligonucleotides (GLP1-CHOP-ASO) intravenously (IV) once per week for 4 weeks, it was expected that the ER stress would be relieved and normalized insulin secretion in the endocrine compartment would be restored.

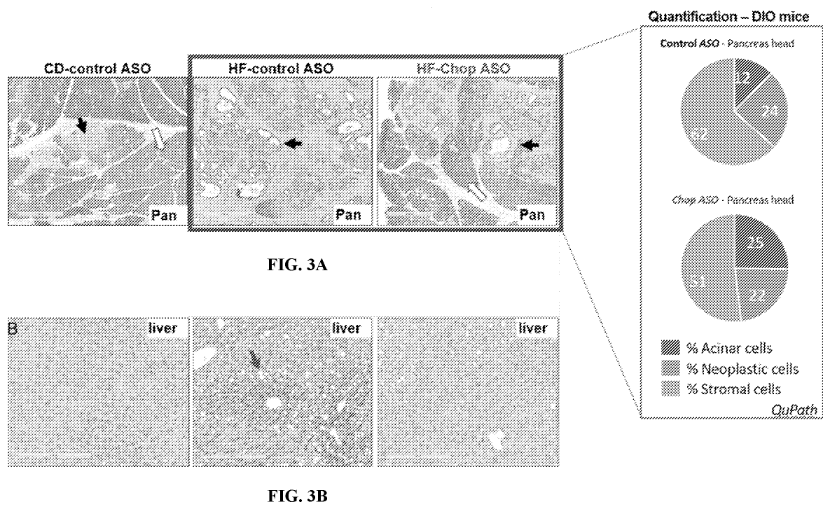

KC mice fed a high-fat diet (HF) develop fatty liver, hyperglycemia and hyperinsulinemia, and become obese. As such, it was found that KC mice that received a HF-diet gained significant weight over the course of the feeding period (FIG. 2D), with CHOP ASO treatment having little impact on body weight. To investigate systemic insulin sensitivity, GTT was performed and an estimation of the Area under the Curve (AUC) of blood glucose levels following glucose administration to fasted mice was determined. KC mice that received HF or CD and treated with control ASO (control treatment; FIGS. 2A and 2C) showed similar fasting blood glucose levels, but glucose levels remained slightly elevated in HF-fed mice during the glucose challenge (FIG. 2A). Conversely, CHOP ASO treatment resulted in improved glucose tolerance (FIG. 2B), with CHOP ASO treated mice displaying lower AUC even for KC mice fed CD (compared to control ASO treated mice); KC mice on the HF-fed that received CHOP ASO treatment displayed significantly lower AUC than KC mice fed CD (FIG. 2C). These data suggest that CHOP silencing in pancreas B-cells improves glucose tolerance in HF-fed KC mice. A comparison of the pancreas and liver histology in KC mice fed HF or CD and treated with GLP1-CHOP ASO compared to CD-fed was determined, (FIG. 3A, left panel); obese KC mice fed HF and treated with control ASO displayed a marked transformation of the pancreas parenchyma—the landscape consisted mainly of neoplastic ducts, cancer cells and extensive stromal areas enriched in immune cells and fibroblasts, normal parenchyma which is rare in tissues of mice fed HF diet alone, because KC mice fed with HF diet have little normal parenchymal tissue in their pancreas. However, GLP1-CHOP ASO reverse the transformation process driven by HF diet and preserved normal pancreas tissue (FIG. 3A, central panel). As expected, mice that received a HF-diet also displayed liver steatosis (FIG. 3B). Importantly, CHOP-ASO treatment in HF-fed mice partially prevented the loss of normal parenchyma and reduced neoplastic duct numbers (FIG. 3A, right panel) and fatty liver (FIG. 3B). These data suggest that as expected, reduction of 3 cell CHOP levels minimized obesity-mediated acceleration of primary tumorigenesis.

Further experiments can be performed such as immunostaining of pancreatic tissue sections and antibodies against CHOP, insulin and markers of epithelial, stromal and immune cells to characterize the effects of the treatments. Furthermore, extent of pancreatic fibrosis can be characterized by Sirius red staining and pancreas and liver steatosis by oil red O staining and by grading the H&E tissue sections to review tumor progression and macro- and micro steatosis to determine whether inhibition of CHOP in β cells reduces fatty liver in KC mice.

In the KC mice model (FIG. 1) that was conducted, a high-fat diet (HF; 45% fat) imposes body weight gain (FIG. 2), fast progression of precancerous lesions (PanINs) and higher PDAC incidence, especially in male mice. It was shown that these HF-diet associated effects were accompanied by marked accumulation in pancreas of tumor-associated fibroblasts, extensive fibrosis, and augmented inflammatory/immune responses (FIG. 3A-B). Moreover, KC mice that received a HF became rapidly obese and developed mild hyperglycemia and hyperinsulinemia, and fatty liver. Importantly, accelerated PanIN and PDAC progression in obese mice appeared regulated, in part, by the high levels of insulin produced in the endocrine compartment of the pancreas and insulin/insulin growth factor 1-R (IGF1-R) signaling.

The transcription factor CHOP is important for insulin folding and secretion in β-cells. As described herein, specific deletion of CHOP in β cells alleviated ER stress, delays glucose-stimulated insulin secretion and abrogated liver steatosis in mice fed a HF diet. As shown, CHOP silencing using antisense oligonucleotides (ASO) conjugated to Glucagon-like peptide 1 (GLP1) to specifically decrease expression of CHOP in β-cells results in normalized insulin production and blood insulin levels and reduced HF-induced fatty liver. These findings identify CHOP inhibition in pancreatic β cells as a therapeutic strategy to alleviate dysregulated insulin secretion and fatty liver disease in diabetes.

### Example 2. Deciphering the Mechanisms Underlying Obesity and Diabetes Promotion of PDAC Tumor Progression

The βKO mouse models (DDIT3 floxed/floxed: RIP-CreERT2) were utilized for this study. Male mice were fed control (CD) or a high-fat diet (HF) starting at 6 weeks of age. After 5 weeks on these diets, mice were treated with ASO conjugated to GLP1 ((GLP1-CHOP-ASO) at a dose of 1 mg/kg, once weekly for 4 weeks) to specifically decrease expression of CHOP in pancreatic β cells. Control mice received control oligonucleotides (control ASO). To gain insight on the effects of the treatments on glycemic control, glucose tolerance tests (GTT) were conducted 4 days after the last ASO administration. Blood, pancreas and liver tissues were collected after the glucose challenge to analyze PanIN and PDAC progression, stromal expansion and the inflammatory/immune response. Similar studies can be carried out using female mice, and such studies can determine whether there may be any gender-specific differences.

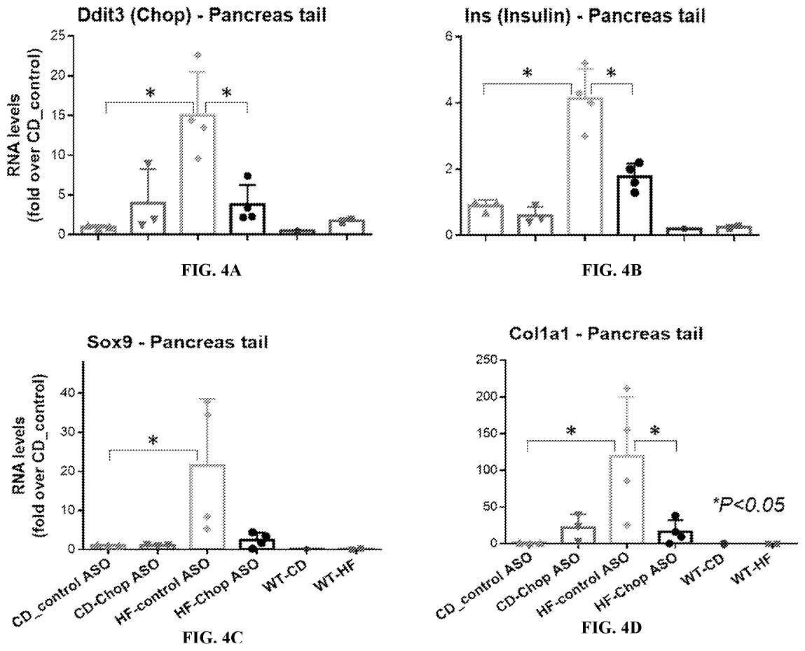

HF diets are associated with obesity and obesity may be associated with aberrant insulin regulation in the pancreas. As described herein, inhibition of CHOP in β cell may lead to the downregulation of insulin expression in the pancreas β cell (FIG. 4). Inhibition of CHOP in β cell as disclosed may lead to reduction in mRNA levels of CHOP and insulin in the pancreas following HF diet or in conditions of obesity. As shown, inhibition of CHOP in β cell by provision of CHOP inhibiting moiety as disclosed herein perturbed abnormal local and systemic insulin production. As described herein, perturbation of abnormal local and systemic insulin production due to administration of a CHOP inhibiting moiety modulated the endocrine compartment of the pancreas and improve, reduce, or slow, or abrogate the initiation or progress of PDAC in obesity.

### Example 3. Effect of GLP1-CHOP ASO Treatment on CHOP and Insulin Expression Levels in the Pancreas

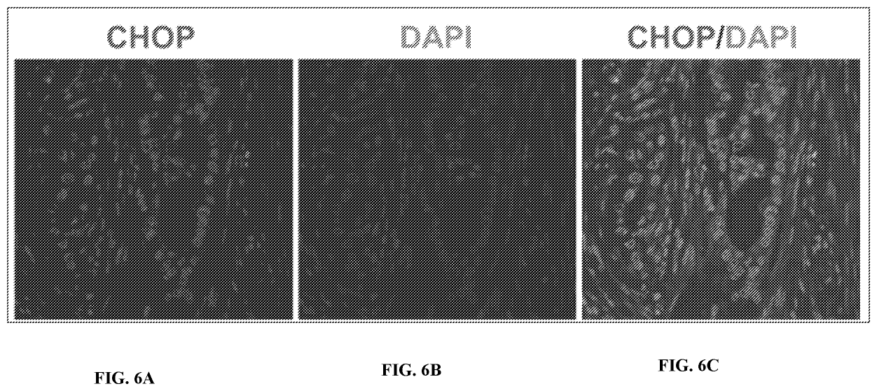

This example illustrates that CHOP expression is upregulated in human PDAC tumors (FIG. 6). Studies were conducted to generate KC mice with specific genetic deletion of CHOP in epithelial cells expressing oncogenic Kras gene. KC mice can be bred with CHOP floxed mice to generate KC;CHOP^{fl/fl} mice.

Studies investigating the consequence of CHOP deletion in PanIN and PDAC initiation and progression using the model of obesity-induced promotion of PDAC and measurements are detailed above.

The effects of a HF diet and pancreatic β cell-specific CHOP genetic deletion on primary tumor growth using syngeneic orthotopic allograft PDAC tumors can be assessed. In these models, tumors developed after injection of mouse neoplastic cells (e.g, isolated from KPC mice) into the normal pancreas of host mice lacking Kras mutations. KPC cells can be orthotopically injected into the pancreas of CHOP βKO (RIP-CreERT;CHOP^{fl/fl}) and littermate control mice (CHOP^{fl/fl}); these mice are randomly allocated to either the control diet (CD) or the high-fat diet (HF). After 8 weeks on diets (or earlier if tumors grow very large), the volume and weight of the allograft tumor are measured and histological analysis of tumor cellularity and stromal expansion, and islet morphology and β cell function are performed.

### Example 4. Examining the Consequences of CHOP Deletion in PanIN and PDAC Initiation and Progression

The effect of GLP1-CHOP ASO treatment in reducing mRNA levels of CHOP (DDIT3) and insulin in the pancreas of HF-fed KC mice was evaluated. To do this, cohorts of KC mice were provided a HF or CD diet (as described above for FIG. 2) then treated with control ASO or with GLP1-CHOP ASO (FIG. 4). Reportedly, KC mice fed HF and treated with control ASO developed significant CHOP (DDIT3; FIG. 4A) and insulin expression in the pancreas (FIG. 4B). However, treatment with GLP1-CHOP ASO effectively reduced CHOP (DDIT3) and insulin expression in KC mice that received the HF diet (FIGS. 4A-4B). Similarly, treatment of mice on a HF diet GLP1-CHOP ASO was associated with significant decreases in mRNA levels of the ductal marker Sox9 and the stromal marker Col1a1 (a collagen chain type highly expressed in PDAC tumors) (FIGS. 4C-4D).

These findings suggest that the endocrine compartment of the pancreas modulates PDAC initiation and progression in obese mice through mechanisms involving abnormal local and systemic insulin production.

### Example 5. Immune Cell Profiling in KC Mice Fed CD and HF by NanoString GeoMx® Digital Spatial Profiling

In this example, investigations explored and quantified the effects of obesity on the tumor microenvironment using a GeoMx Digital Spatial profiling. To do this, pancreas tissues from KC mice fed CD and HF for 2 and 5 months (3 mice per group) were analyzed. As stated, a GeoMx® Immune Cell Profiling, GeoMx® Immune Cell Typing and GeoMx® were utilized to assess immune activation in status panels that included a total of 35 markers of stromal cells, immune cells, immune cell activation and immune checkpoints. A total of 3 regions of interest in each tissue: normal parenchyma, enriched-PanIN areas and enriched-stromal areas were analyzed.

### Results

The data obtained show that obesity associated with HF markedly altered the pancreas landscape in KC mice by significantly expanding the PanIN and stromal areas and reducing the normal parenchyma. As such, the results indicate HF diet promotes the accumulation of myofibroblasts and type 1 macrophages mainly in stromal areas, while immunosuppressor type 2 macrophages predominate in areas surrounding PanINs. Consistent with these data, cytotoxic T lymphocytes were sparse in HF-fed mice and were found mainly in stromal areas and were poorly represented in areas enriched in PanINs or cancer cells. The data indicates that obesity promotes an immunosuppressive microenvironment that is permissive of PDAC development and tumor progression.

A GeoMx Spatial profiling or CyTOF can be employed to determine the effects of CHOP silencing in obese KC mice on immune responses in PDAC tumors and liver metastasis.

### Example 6. Examining Whether β Cell Dysfunction Promotes Pancreatic Cancer Metastasis to the Liver

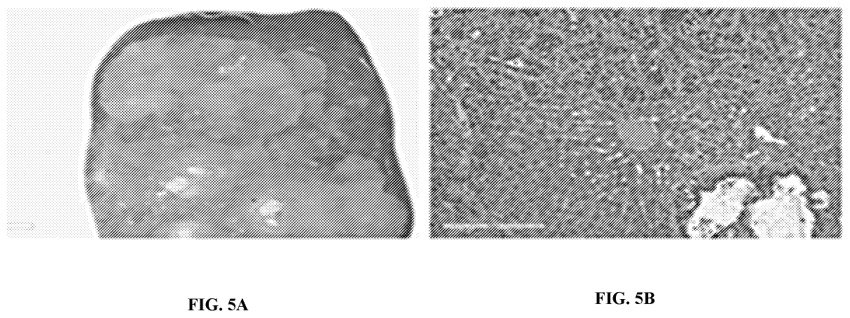

This study was conducted to establish syngeneic orthotopic model of liver metastasis by intrasplenic injection of pancreatic KPC cells into CHOP βKO and control littermate mice with no CHOP deletion. Both groups of mice were morbidly sick 12d post KPC cell injection, due to rapid metastasis of KPC cells into liver, following the standard 1 million KPC cells transplant (FIG. 5). The animal study was performed with inhibition of CHOP specifically in β cells (CHOP ASO administration) when mice were fed a HF diet. Data analysis and tissue characterization is ongoing. Syngeneic orthotopic model of liver metastasis have been established by intrasplenically injecting pancreatic KPC cells into the obese CHOP βKO mice. Data from these studies further demonstrate tumor engraftment and liver metastasis.

To avoid getting mice morbidly sick (FIG. 5) when orthotopic model of liver metastasis was being generated, the process of cell dose optimization was used instead to achieve controlled liver metastasis. The process of cell dose optimization utilized at least 1×10,000 to 1×25,000 cells per mouse per splenic injection, or about 1×10,000 to 1×25,000 cells per mouse per splenic injection, or less than 1×10,000 cells per mouse per splenic injection, or less than 25,000 cells per mouse per splenic injection or more than 25,000 cells per mouse per splenic injection.

### Example 7. Determining Whether CHOP Deletion in β Cells can Reduce Liver Metastasis

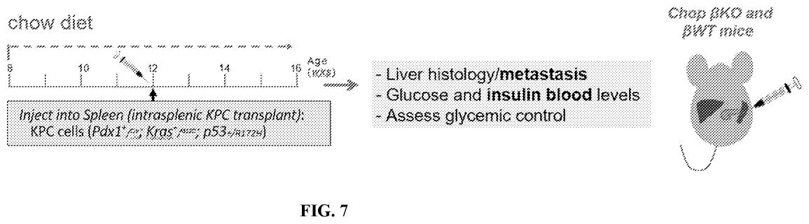

To examine whether CHOP deletion in β cells can reduce liver metastasis, a syngeneic orthotopic model of liver metastasis was established by intrasplenically injecting pancreatic KPC cells into CHOP βKO and control mice. This mouse model rapidly develops metastasis, bypassing early steps involving primary tumors (FIG. 7). The control and CHOP βKO mice were injected with 10⁶ cells per mouse and the tumors showed rapid formation in all the mice. The controlled metastasis can be established in which optimization steps can ensure fewer cells are injected. Following the injection and establishment of controlled metastasis, 8-10-week-old CHOP βKO and control mice were allocated to feed on either a high-fat diet (HF) or a control diet (CD) for 3 months prior to splenic KPC cell injections. The metastatic tumor nodules in liver and other metastatic sites were characterized in mice on diets by comparing the number and size of liver tumor nodules in HF-fed CHOP βKO and control mice to determine whether hyperinsulinemia and associated β cells dysfunction promoted cancer cell metastasis. Measurements were taken to measure liver weight, number and size of tumors as well as liver histology to assess hepatic steatosis, tumor characteristics and features of fibroinflammatory responses.

### Results

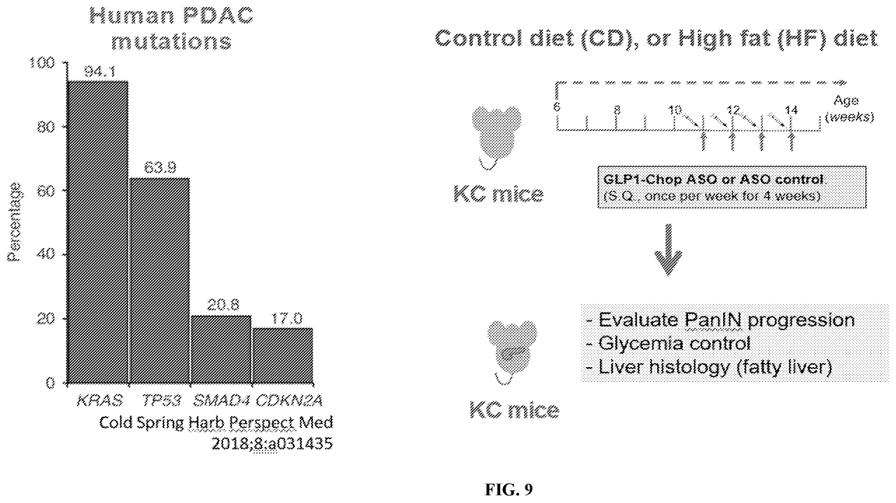

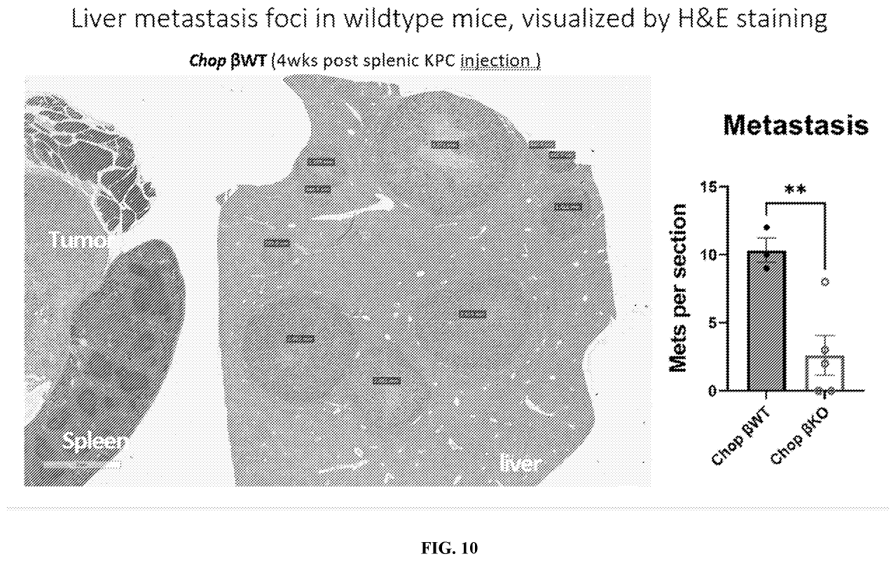

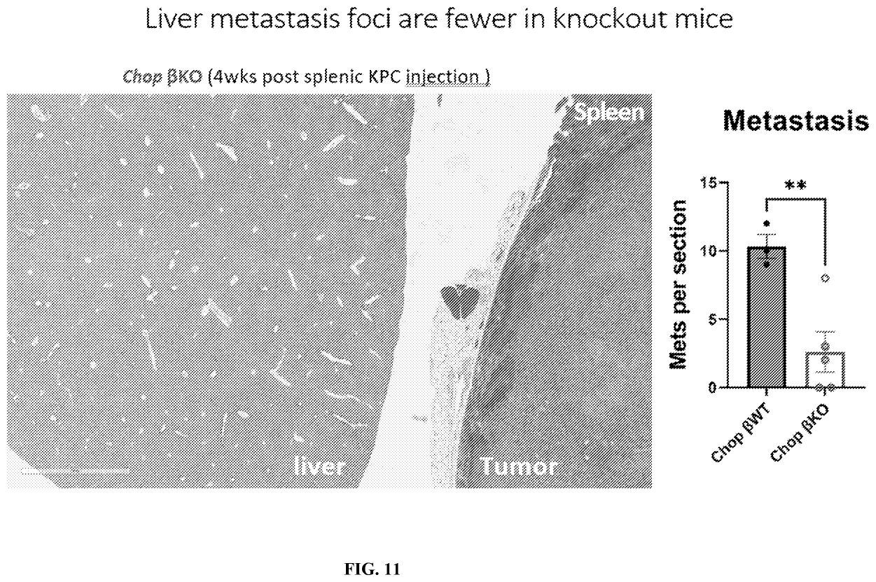

The metastatic tumor nodules in liver and other metastatic sites characterized in mice on diets by comparing the number and size of liver tumor nodules in HF-fed CHOP βKO and control mice indicated a significantly higher volume and large tumors in HF-fed control mice than HF-fed CHOP βKO (FIG. 9) and the same was true for metastasis to the liver and other sites (FIG. 10). These studies determined that hyperinsulinemia and associated β cells dysfunction promoted cancer cell metastasis. Essentially, the studies suggested that: in β cells, the unfolded protein response (UPR) regulates the efficiency of proinsulin processing and secretion based on demand. In the obese cases, high insulin production by β cells leads to hyperinsulinemia, which drives hepatic steatosis.

These investigations can address whether (1) mouse models of HF-driven PDAC promotion of hepatic steatosis could favor production of pro-fibroinflammatory factors by liver cells and high levels of glucose and other growth factors systemically to influence primary PDAC tumor growth, or whether they (2) can form a niche favorable to metastasis or exosomes from the primary tumor. The impetus for (1) and (2) studies above stems from the prior results disclosed herein in which the inventor showed that CHOP deletion in pancreatic β-cells of mice corrects hyperinsulinemia and hepatic steatosis. Patients also exhibit systemic and hepatic hyperinsulinemia.

### Example 8. Exemplary Experiment to Identify Misfolded Protein Events Leading to ER Stress and CHOP Upregulation in Cells Expressing Oncogenic Kras

The upregulation of CHOP observed in PDAC tumors (FIGS. 4 and 6) is likely associated with ER protein misfolding (including insulin misfolding). Here, the study can use BiP-Flag mice (mice strains available in the laboratory) to identify protein misfolding events during obesity-induced tumor growth. BiP (Grp78) is an ER chaperone that assist with protein folding within the ER by directly binding to misfolding proteins. BiP-Flag mice express BiP with a Tag modification of the endogenous BiP allele. BiP-Flag mice allow for cell-type specific efficient pulldown and recognition of misfolded BiP-client proteins. BiP-Flag mice can be bred with KC mice to generate BiP-Flag KC mice. A cohort of these mice can be fed CD or HF to promote PDAC progression and PDAC tumors cab be analyzed using experimental approaches described. To identify proteins that misfold and bind to BiP, tumor proteins can be subjected to anti-Flag IP and identified by Mass Spec.

### Mouse Strains:

Some of the strains, methodology, disclosures utilized, discussed or disclosed herein are shown Example 9, Table 1 and in any additional supplementary information herein disclosed with this application including published articles of the inventors (Peng et al., BioRxiv Oct. 9, 2021; Yong et al., Sci Trans Med 2021, 13; Zhou et al., Circ Res. 2015; and Yong et al., Pancreas, Vol 51, Number 7, August 2022, herein disclosed with this filing).

Both the Peng et al. and the Yong et al., references shown here are hereby incorporated by reference to this disclosure, in their entirety.

### Example 9. Investigation of the Relationship Between Exocrine and Endocrine Compartments in PDAC

Studies to unveil mechanisms of obesity and diabetes that promote PDAC tumor progression and metastasis to the liver were undertaken.

Previously developed animal models and cell lines (in Example 11 and Table 1) were utilized in a new approach to investigate how the endocrine compartment of the pancreas, and in particular, the pancreatic β cell, influences PDAC development, tumor growth and metastasis to the liver. Mouse models of obesity induced PDAC promotion with or without CHOP deletion in β cells can be assessed to study the specific effects of hyperinsulinemia and hepatic steatosis (and pancreatic steatosis) on primary tumor growth and stromal expansion and on the number and growth of liver metastases. By normalizing insulin secretion and glycemic control in obese mice, it is possible to determine those cancer promoting effects of obesity that are dependent and independent of β cell dysfunction and gain molecular-mechanistic insights required to formulate effective PDAC-preventive strategies.

### Example 10. Mechanisms Underlying Obesity and Diabetes Promotion of PDAC Tumor Progression and Metastasis to the Liver

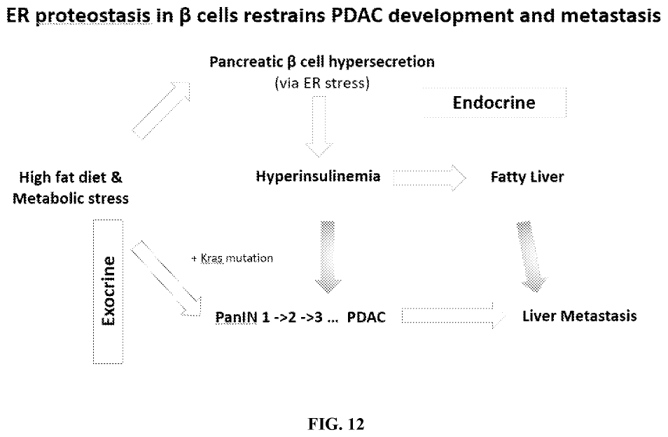

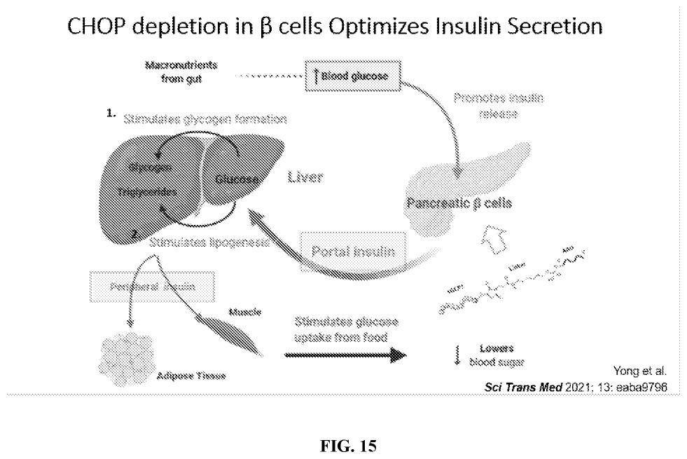

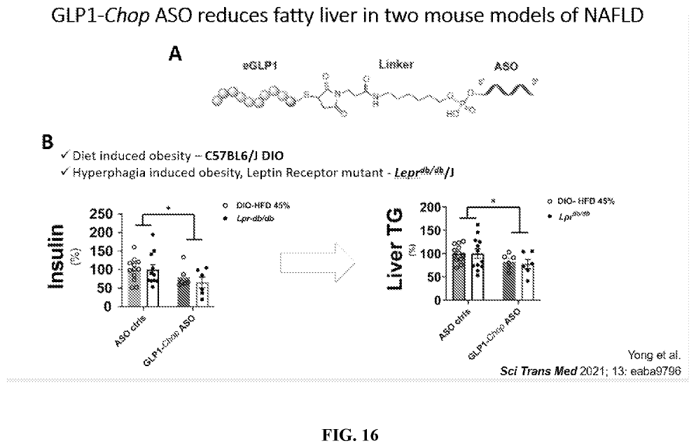

The mechanisms underlying promotion of PDAC with obesity and T2DM are beginning to be understood (FIG. 1, FIG. 12 and FIG. 15); emerging evidence implies that the endocrine and exocrine pancreatic compartments interact during PDAC progression. Diet-induced obesity accelerated PDAC progression and invasion in orthotopic and genetic mouse models of PDAC and the anti-diabetic drug metformin reduces PDAC incidence in these models. The studies disclosed herein have characterized a model of diet-induced obesity in mice carrying a pancreas-specific oncogenic Kras mutation (P48^{+/Cre};Kras^{+/G12D}; KC). As disclosed herein, a high-fat diet (HF; 45% fat) imposes body weight gain, fast progression of precancerous lesions (PanINs) and higher PDAC incidence. The developing lesions and PDAC exhibit a robust tumor microenvironment with extensive associated myofibroblasts, fibrosis, and immune cell infiltration. HF also facilitates metastasis in Kras mice, preferentially to the liver. Obese KC mice also exhibit fatty liver, hyperinsulinemia and hyperglycemia. To identify molecular biomarkers of obesity induced PDAC promotion, proteomic analysis of tumor tissues from KC mice fed CD and HF were performed.

### Results

Key findings from this study show, compared to lean mice, obese mice showed (1) enhanced levels of matrisomal proteins (e.g., osteopontin, tenascin-C, SPARC-like protein 1 and S100A11) that support tumor growth and cancer cell invasion and (2) decreases in factors required to maintain the secretory phenotype of normal parenchymal cells including the transcription factor X-Box Binding Protein 1 (XBP1; Z activation score: −5.0, obese vs. lean mice). XBP1 regulates many genes involved in endoplasmic reticulum (ER) function and the Unfolded Protein Response (UPR). XBP1 deficiency in acinar cells leads to pancreas inflammation and facilitates tumorigenesis and metastasis in KC and KPC (Pdx1^{+/Cre};Kras^{+/G12D}; p53^{+/R172H}) mice.

The mechanisms whereby obesity (and/or diabetes) promotes PDAC growth in the mouse models are likely multifactorial. The systemic and local inflammation in pancreas and adipose tissue of obese mice appear conducive to PDAC promotion and desmoplasia. Also, high levels of local and circulating insulin and insulin-like growth factor1 (IGF-1) in obese mice support growth of cancer and stromal cells.

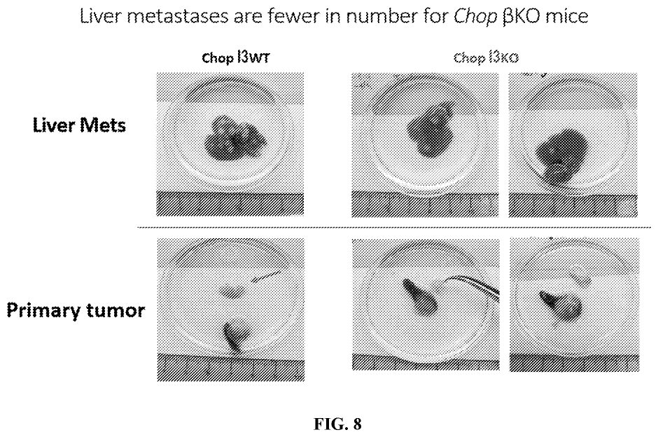

Patients with type 2 diabetes mellitus (T2DM) display extensive stroma with active myofibroblasts in islets and peri-islet areas. Obesity may also cause metabolic reprogramming in neoplastic and stromal cells. Diet-induced obesity is also linked to fatty liver disorders. The relationship between fatty liver and PDAC progression and metastasis is unclear. Although poorly investigated in PDAC, fatty liver disease can be associated with higher number of liver metastases and higher risk of recurrence in colorectal and breast cancer. Thus, it is conceivable that fatty liver may favor PDAC metastasis to the liver and to other organs (FIG. 7 and FIG. 8).

TABLE 1
Key Resources
Experimental Models: Organisms/Strains

| | | |
|---|---|---|
| Mouse, CHOP gene floxed | The Jackson Laboratory | #030816, |
| | | B6.Cg-DDIT3tm1.1Irt/J |
| Mouse, RIP-CreERT2 transgenic | The Jackson Laboratory | #008122, |
| | | Tg(Ins2 |
| | | cre/ERT)1Dam/J |
| KC mouse, p48^{+/Cre}: LSL- | See reference | PMID: 14706336 |
| KRAS^{G12D} | | |
| Mouse, CHOP gene floxed: RIP- | Bred in house | |
| CreERT2 | | |
| Mouse, BiP-Flag tagged | Bred in house | |

Cell lines

| | |
|---|---|
| KPC cells | From Dr. Stephen Pandol's lab |

TABLE 2
Exemplary Primers Used in the Present Disclosure

| Name | Sequence | Used for |
|---|---|---|
| P2093_41 | CTAACCTATTCCTGGTAAGTGGTATCCG (SEQ ID | Targeting vector |
| | NO: 1) | construction |
| P2093_51 | TAAGCATTGGTAAGACGTCAAGCCCCTCTGACC | Targeting vector |
| | TTGTATTAC (SEQ ID NO: 2) | construction |
| P2093_44 | TAAGCATTGGTAAGCGGCCGCGTGCACTGATCT | Targeting vector |
| | GCTAGAGCTG (SEQ ID NO: 3) | construction |
| P2093_54 | CTAATGAACACAGAAGGGGAGGTTTATG (SEQ | Targeting vector |
| | ID NO: 4) | construction |
| 2093_Lo5WT_ | GAGGGGCTTACAATGCTTTG (SEQ ID NO: 5) | 5′ end Hspa5 wt allele |
| F | | validation |
| 2093_Lo5WT_ | GGGTCGTTCACCTAGAGTAAG (SEQ ID NO: 6) | 5′ end Hspa5 wt allele |
| R | | validation |
| 2093_Lo9WT_ | AAGAGCAGTAGCACCCAGTGAGTT (SEQ ID NO: | 5′ end Hspa5 wt allele |
| Probe | 7) | validation |
| 2093_LoWT3_ | AGATCAGTGCACCTACAA (SEQ ID NO: 8) | 3′ end Hspa5 wt allele |
| F | | validation |
| 2093_LoWT3_ | CAGGATGCGGACATTGAA (SEQ ID NO: 9) | 3′ end Hspa5 wt allele |
| R | | validation |
| 2093_LoWT3_ | AGCAAACTCTATGGAAGTGGAGGCC (SEQ ID | 3′ end Hspa5 wt allele |
| Probe | NO: 10) | validation |
| 1.638_goNoz_ | CTTCTTGACGAGTTCTTCTGAGG (SEQ ID NO: 11) | Gain of Neo validation |
| F | | |
| 1.638_goNm_ | AACAACAGATGGCTGGCA (SEQ ID NO: 12) | Gain of Neo validation |
| R | | |
| 1.63830Nm_ | TCAGCCTCGACTGTGCCTTCTAGT (SEQ ID NO: | Gain of Neo validation |
| Probe | 13) | |
| 1.638_ | ATTGAGGAGTGGCAGCATATAG (SEQ ID NO: 14) | Gain of Flp validation |
| goFlpOz_ | | |
| F | | |
| 1.6383oFlpOz_ | GGTAGTCTAGTACCTCCTGTGATA (SEQ ID NO: | Gain of Flp validation |
| R | 15) | |
| 1.638_ | TGCTTCCTTCAGCACTACCCTTTAGC (SEQ ID NO: | Gain of Flp validation |
| goFlpOz_ | 16) | |
| Probe | | |
| 2093 | GATTCAGTAGACCGCTGTTGG (SEQ ID NO: 17) | Gain of 2093_Teak conKI |
| GoConK_F | | allele validation |
| 2093 | GCACTGGGATCCCTACATTAAC (SEQ ID NO: 18) | Gain of 2093_Teak conKI |
| GoConK_R | | allele validation |
| 2093 | TGAAATTAAGAGCAGTAGCACCCAGTGA (SEQ | Gain of 2093_Teak conKI |
| GoConK_ | ID NO: 19) | allele validation |
| Probe | | |
| 2093 GoK_F | TATCCTCCCAGGGAAAGA (SEQ ID NO: 20) | Gain of 2093_Teak conKI |
| | | allele validation |
| 2093 GoK_R | AGCATTGTTCTAGACCGC (SEQ ID NO: 21) | Gain of 2093_Teak conKI |
| | | allele validation |
| 2093 GoK | AAGCCCCTCTGACCTTGTATTAC (SEQ ID NO: 22) | Gain of 2093_Teak conKI |
| Probe | | allele validation |

### Example 11. Generation of Mouse Strains—Materials and Methods

Generation and genotyping of CHOP^{Fe/Fe} (DDIT3^{Fe/Fe}) mice: The transgenic mice were generated in house, and characterized as previously published (1). This line is donated to The Jackson Laboratory, with strain catalogue #030816 (“B6.Cg-DDIT3^{tm1.11rt/J}”). For genotyping of the mice carrying the foxed CHOP/DDIT3 allele, primers targeting the L83 element were used, as described on the Jackson Laboratory website (www.jax.org/strain/030816).

Breeding strategy to generate the CHOP β-KO mice: CHOP^{Fe/Fe} mice were first bred with a RIP-CreER transgenic mouse in which the fusion protein of Cre recombinase and a mutant human Estrogen Receptor (ERT2) is driven by the rat insulin II (Ins2) promoter (2) to generate the CHOP^{+/Fe}: RIP-Cre founders. The CHOP^{+/Fe}: RIP-Cre mice were further bred with CHOP^{+/Fe} mice to generate littermates of the following genotypes: 1). CHOP^{Fe/Fe}: RIP-Cre; 2). CHOP^{+/Fe}. RIP-Cre; 3). CHOP^{+/+}: RIP-Cre. After TAM injections, specifically in the β-cells, the DDIT3 loci became: 1). CHOP^{Δ/Δ}: RIP-Cre; 2). CHOP^{+/Δ}: RIP-Cre; 3). CHOP^{+/+}: RIP-Cre respectively. In selected experiments, CHOP^{-/-} mice were used (instead of CHOP^{+/Fe} mice) to breed with the CHOP^{+/Fe}: RIP-Cre founder so that progeny of the following genotypes can be generated: 1). CHOP^{Fe/-}: RIP-Cre; 2). CHOP^{+/-}: RIP-Cre. After TAM injections, specifically in the β-cells, the DDIT3 loci became: 1). CHOP^{Δ/-}: RIP-Cre; 2). CHOP^{+/-}: RIP-Cre; respectively.

Mouse diet and husbandry: Standard mouse diet (Teklad Global Irradiated Diet #T.2918.15) and water were available ad libitum at all times, except for a 4 to 6-h fasting period before intraperitoneal glucose tolerance test (IPGTT) when indicated. For the high-fat diet studies (HFD, 45% fat in Kcal %, Open Source Diets #D12451), male mice from 2-3 litters were mixed and matched, with n=2-4 mice per cage without genotype separation. Mice were only separated into individual cages for food intake measurement, when indicated. All procedures were performed by protocols and guidelines approved by the Institutional Animal Care and Use Committee (IACUC) at the SBP Medical Discovery Institute (AUF 17-066).

Tamoxifen injections: Deletion of the floxed CHOP allele in pancreatic β-cells was induced by intraperitoneal injection of tamoxifen (TAM, Sigma Cat #T5648) dissolved in 10% ethanol in PBS in the dose of 2 mg per injection. Four to five doses of 4-10 mg TAM were administered over 2 weeks to adult mice (2, 3).

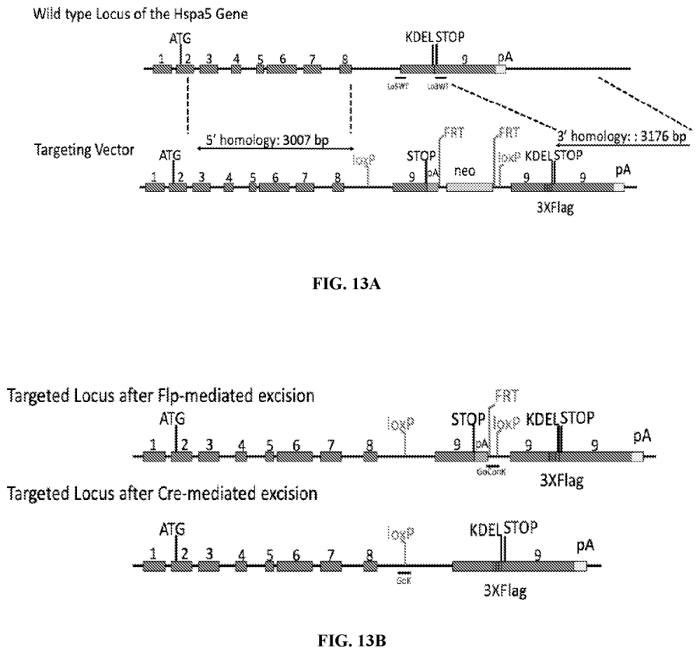

Generation of BiP-3×Flag mice: (Peng Y et al., 2021 Epitope-tagging of the endogenous murine BiP/GRP78/Hspa5 locus allows direct analysis of the BiP interactome and protein misfolding in vivo, the entirety of which is hereby incorporated by reference in this disclosure). A conditional knock-in mouse model was generated by modifying of the Hspa5 locus. This was achieved by floxing a targeted WT exon 9-pA cassette upstream of the knock-in exon 9 where a 3×FLAG sequence was introduced immediately prior to the KDEL ER retention signal (“KDEL” disclosed as SEQ ID NO: 23). Additionally, an FRT-flanked neomycin cassette was introduced into the floxed region. The genetic modification was introduced into Bruce4 C57BL/6J ES cells (14) via gene targeting. Correctly targeted ES cell clones were identified and then injected into goGermline blastocysts (15, 16). Male goGermline mice were bred to C57BL/6J females to establish heterozygous germline offspring on a C57BL/6J background. a). Vector construction. A replacement vector targeting Hspa5 exon 9 coding sequence region (CDS region) was generated by assembly of 4(ABCD) fragments using sequential cloning. The first fragment which encompassed the 3 kb 5′-homology arm was generated by PCR amplified from C57BL/6J genomic DNA using primers P2093_41 and P2093_51. The second and third fragments which comprise loxP-exon9-BGHpA and exon 9-3×Flag were synthesized by Genewiz, respectively. The fourth fragment comprising the 3.2 kb 3′-homology arm was generated by PCR amplification from C57BL/6J genomic DNA using primers P2093_44 and P2093_54. Synthesized fragments and PCR primers used to amplify the fragments included all the restriction enzyme sites required to join them together and to ligate them into the Surf2 vector backbone (Ozgene). The final targeting vector 2093_Teak_ABCD contained an FRT-flanked neomycin selection (neo) cassette, an exon 9 coding sequence sequentially with an inserted bovine growth hormone (BGH) polyA tail, an additional exon 9 coding sequence sequentially with a 3×FLAG tag cassette right before the KDEL sequence (SEQ ID NO: 23), 5′- and 3′-loxP sites (FIG. 13A). For sequence information of the primers see Table 2. The targeting vector was entirely sequenced and then linearized by digestion with PmeI before electroporation into C57BL/6J Bruce4 ES cells (14). Neo-resistant ES cell clones were screened by qPCR to identify potentially targeted clones. b). Targeting murine ES cells through homologous recombination. TaqMan® copy number reference assays were used to measure copy number in the genome. Two pairs of primers were used to amplify the WT locus at the extreme 5′ and 3′positions to detect 2 copies from the WT allele and 1 copy from the targeted allele (primers, 2093_Lo5WT and 2093_LoWT3). Another primer pair targeting Neo sequence was used to test the targeted allele (primer, 1638_goNoz). Two genes from Y chromosome (1 copy) and chromosome 8 (2 copy) were used as control. Two positive clones, Clones I_1D08 and I_1G08, were confirmed as correctly targeted and were used to injection into goGermline blastocysts. c).

Production of mice heterozygous for a BiP-FLAG allele: ES cells from clones I_1D08 and I_1G08 were injected into goGermline donor blastocysts to generate chimeras. A total of 84 injected blastocysts were transferred into 7 recipient hosts. These resulted in 35 offspring, of which 28 were male chimeras. Four males were chosen for mating with homozygous Flp mice. A total of 17 pups was born from three litters, including 10 WT and 7 WT/conKI (FIG. 13B).

### Example 12: CHOP Deletion in Cells Prevents Liver Triglyceride Accumulation in Male DIO Mice

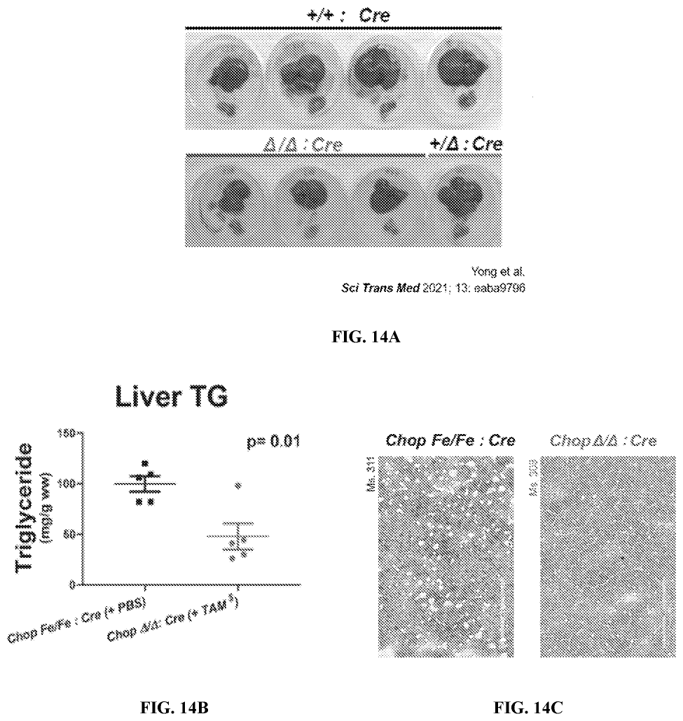

Because diet-induced obesity (DIO) is a known risk factor for NAFLD, CHOP-KO in β cells were tested to determine whether they could correct DIO-induced NAFLD. Mice were challenged with a high-fat diet (HFD; 45% fat in kcal) for 20 weeks, starting at 9 weeks of age, with CHOP deletion induced by TAM at 10 weeks of HFD feeding. Littermates harboring WT CHOP alleles were selected as a TAM-treated control group. Before CHOP deletion, the two groups were metabolically indistinguishable with no significant difference in body weight or blood glucose concentrations, either before or after 10 weeks of HFD. After CHOP deletion, a moderate but significant decrease in body weight was observed in CHOP βKO mice (P<0.01 at week 20), which had no effect on blood glucose. After 20 weeks of HFD feeding, murine livers were dissected for visual inspection and liver TG analysis. HFD feeding caused hepatomegaly and liver discoloration associated with fatty deposits in control littermates as well as for a Chop β heterozygous (βHet) mouse in the litter (FIG. 14A). In contrast, the three CHOP βKO mice had normal-sized livers and appeared healthy (FIG. 14A). The morphological impression was further confirmed quantitatively by significantly reduced liver weight and TG content (FIG. 14B) in CHOP βKO mice compared to littermates. Chronically reduced β cell insulin secretion may prevent fatty liver development in the HFD-fed C57BL/6 mice.

### Example 13: Glucose Clamp Study on DIO Mice Indicate CHOP Deletion in β Cells Improves Insulin Secretion

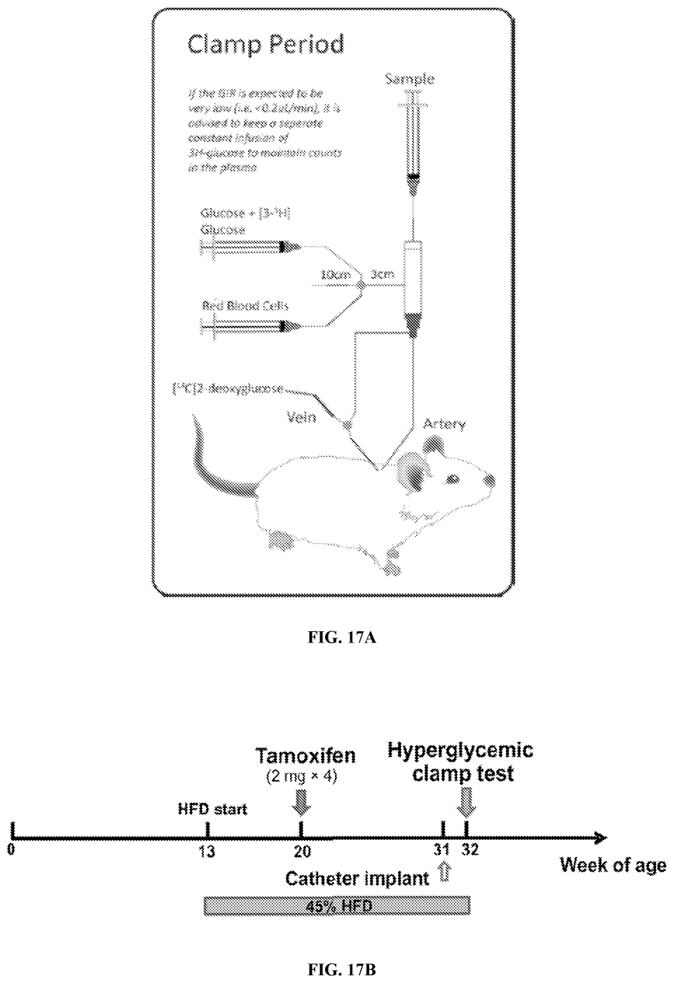

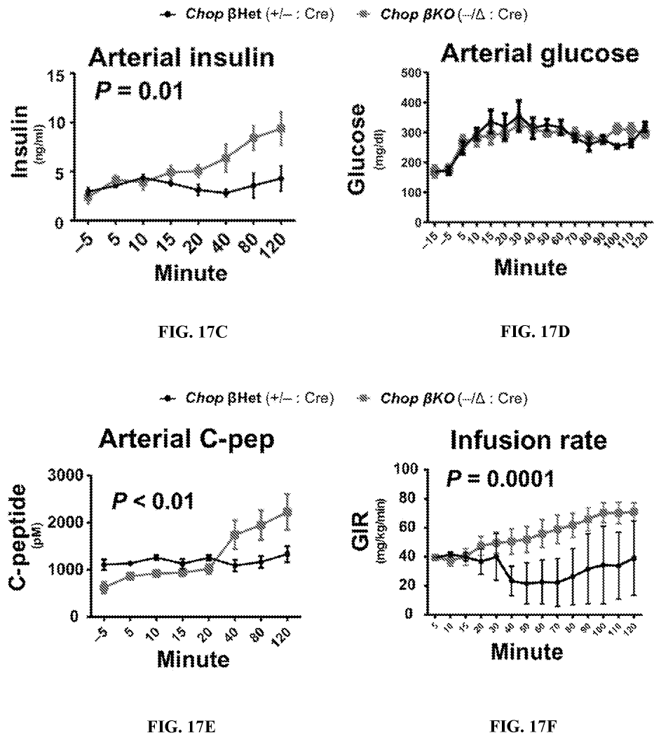

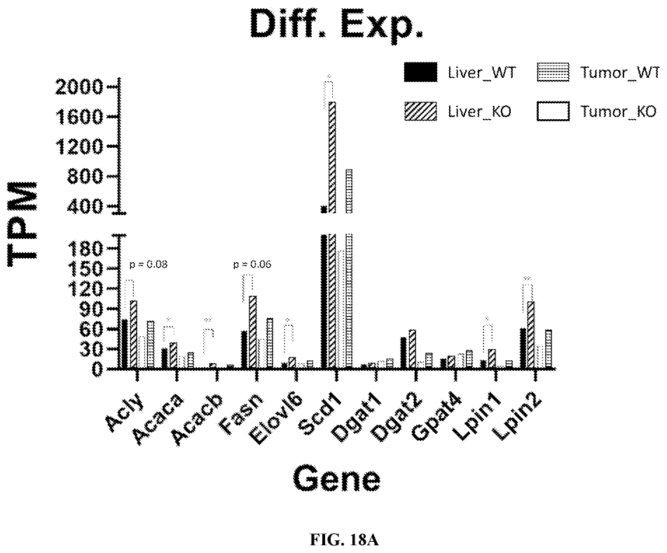

In another experiment, a glucose clamp study was conducted to examine the effect of CHOP deletion on insulin secretion in mouse β cells (FIG. 17A). Briefly, mice were divided into two groups: one group comprised mice expressing endogenous CHOP in β cells (CHOP βHet (+/−: Cre)), and the other comprised mice with CHOP knocked out in 1 cells (CHOP βKO (−/Δ: Cre)). Symbols in parenthesis represent CHOP gene status. The negative symbol (“−”) is indicative of a knockout allele of the CHOP gene. The delta symbol (“Δ”) is indicative of a foxed CHOP gene with exon 3 deleted by Cre/ERT recombinase after tamoxifen injection. All Cre constructs comprise a rat insulin promoter-driven Cre/ERT fusion gene (RIP-Cre/ERT) and both groups of mice express the RIP-Cre/ERT gene in their β cells. All mice in this study were fed a 45% high-fat diet starting at 13 weeks of age and terminating at 32 weeks of age. At 20 weeks of age, or equivalent to 7 weeks after the initiation of the high-fat diet, mice were administered 2 mg Tamoxifen at regular intervals of once every two days for a total of four times. The catheter was implanted at 31 weeks of age and the hyperglycemic clamp test began at 32 weeks of age (FIG. 17B).

Mice in both groups were catheterized at the carotid artery and jugular vein, the former of which comprised two lines: one line for radiolabeled glucose (Glucose+[3-³H]) and the other for red blood cells (FIG. 17A). Glucose was delivered at a glucose infusion rate between around 40 and around 60 mg/kg/min in CHOP βKO (−/Δ: Cre) mice, and between around 20 and around 40 mg/kg/min in CHOP βHet (+/−: Cre) mice (FIG. 17F). Carbon-14-labeled 2-deoxyglucose ([¹⁴C] 2-deoxyglucose) was administered through the jugular vein catheter and blood samples taken from the carotid artery. Concentrations of arterial insulin and arterial glucose were measured in both groups of mice starting at approximately five to fifteen minutes prior to the initiation of the flow of the two types of aforementioned radiolabeled glucose and red blood cells, and measurements were stopped at about 120 minutes.

Both CHOP βHet (+/−: Cre) and CHOP βKO (−/Δ: Cre) mice expressed a similar trend of arterial glucose concentration throughout the course of this study (FIG. 17D). Five to fifteen minutes prior to initiation of the flow of the two types of aforementioned radiolabeled glucose and red blood cells, arterial glucose was at its nadir in both groups of mice, measuring at a little less than 200 mg/dl. At their height, the concentrations of arterial glucose for both groups of mice measured at around 325-350 mg/dl.

While the pattern of arterial glucose was similar in both groups of mice, CHOP βHet (+/−: Cre) and CHOP βKO (−/Δ: Cre) mice exhibited different arterial insulin profiles (FIG. 17C). Measurements of insulin in both groups of mice measured at around 3 ng/ml approximately five minutes prior to initiation of the flow of the two types of radiolabeled glucose and red blood cells. The concentration of arterial insulin of CHOP βKO (−/Δ: Cre) mice did not fluctuate significantly throughout the course of the clamp study, remaining below approximately 5 ng/ml; however, a positive trend was observed in the arterial insulin profile of CHOP βHet (+/−: Cre) mice such that insulin increased over the course of the 120 minutes after initiation of radiolabeled glucose and red blood cell flow. At the 120-minute timepoint, CHOP βKO (−/Δ: Cre) mice had higher levels of insulin (about 10 ng/ml) compared to CHOP βHet (+/−: Cre) mice (about 5 ng/ml).

Additionally, arterial C-peptide (c-pep), which is synthesized when insulin is produced and released, was measured during the glucose clamp study (FIG. 17E). CHOP βHet (+/−: Cre) were observed to have insignificant fluctuations in arterial C-peptide concentration, hovering at a little above 1000 pM of C-peptide over the course of the 120-minute study. CHOP βKO (−/Δ: Cre) mice were observed to experience an increase in arterial concentration of C-peptide between around 20 and 40 minutes after initiation of the two types of aforementioned radiolabeled glucose and red blood cell flow. Arterial C-peptide concentrations CHOP βKO (−/Δ: Cre) mice were measured at approximately 2200 pM 120 minutes after initiation of the glucose clamp study.

The control CHOP βHet mice showed basal hyperinsulinemia and reduced first-phase responses to glucose (FIGS. 17C and 17E), demonstrating that the HFD model properly replicated the phenotype of humans during the prediabetic and early T2D phases. CHOP βKO mice required a significantly higher glucose infusion rate (GIR) to maintain their blood glucose target at ˜300 mg/dl (=16.7 mM; FIG. 17D) (P=0.0001; FIG. 17F), suggesting that the increased glucose clearance was due to increased insulin secretion and not altered insulin sensitivity.

### Example 14: Upregulation of Liver DNL Pathway is not Due to Increased Insulin in Circulation in CHOP βKO Mice

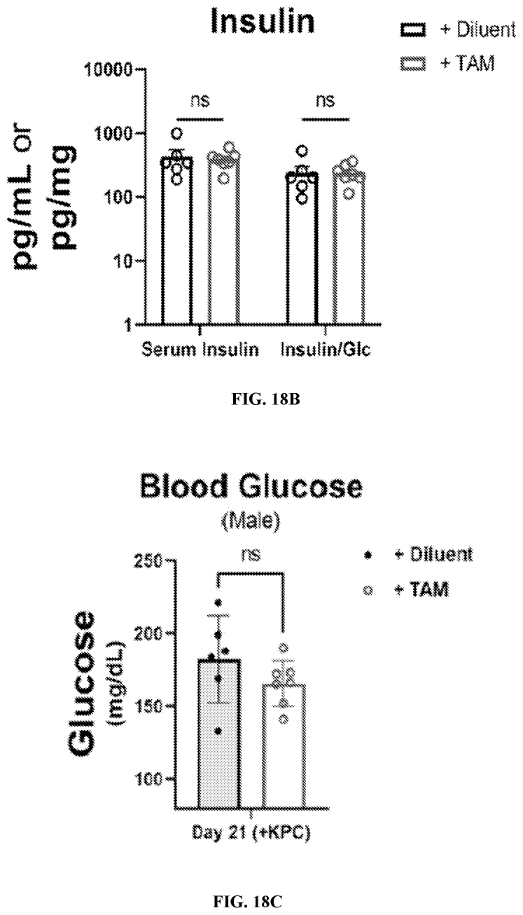

A subset of genes was evaluated for transcript expression levels read as transcripts per million (TPM) indicative of relative gene expression levels (FIG. 18A). Mouse tissue from normal livers expressing wild-type CHOP in β cells (“Liver WT” or “CHOP βHet”) and knocked-out CHOP in β cells (“Liver_KO” or “CHOP βKO”), in addition to mouse tissue from PDAC tumors with wild-type CHOP (“Tumor_WT”) and knocked-out CHOP (“Tumor_KO”) were examined for differential gene expression.

The Scd1 (stearoyl CoA desaturase 1) gene, which encodes the SCD1 liver protein associated with fatty liver, was the most highly expressed gene across all mouse tissues in the subset of genes examined in the present study. The presence of SCD1 protein has been associated with hepatic de novo lipogenesis (DNL); specifically, SCD1's ability to desaturate fatty acids prevents the deleterious effects of increased hepatic de novo lipogenesis (DNL), which generates saturated fatty acids that exert lipotoxic effects and lead to liver fat accumulation. High Scd1 gene expression provides an indicator of upregulation of the liver DNL pathway.

The CHOP βKO normal mouse liver tissue expressed approximately 1,800 transcripts per million of the Scd1 gene, CHOP βHet normal mouse liver tissue expressed approximately 400 transcripts per million of the Scd1 gene, PDAC tumor tissue with wild-type CHOP expressed approximately 180 TPM, and PDAC tumor tissue with knocked-out CHOP expressed around 900 TPM. In addition to the Scd1 gene, the Acly, Fasn, Dgat2, Lpin1, and Lpin2 genes were also generally expressed at higher levels in CHOP βKO normal mouse liver tissue relative to the three other types of mouse tissues studied.

Insulin and glucose concentrations in the sera of fed mice were also measured by Luminex assays (FIGS. 18B-18C). Serum insulin concentrations of both diluent and tamoxifen (TAM)-treated mice were approximately 500 μg/ml or 500 pg/mg. Negligible difference was found for insulin concentrations in the sera of mice treated with diluent and mice treated with tamoxifen; similarly, negligible difference was observed for sera insulin versus sera glucose concentrations between diluent and tamoxifen (TAM)-treated mice (FIG. 18B). Blood glucose concentrations in male mice were about 180 mg/dl in mice treated with diluent and approximately 160 mg/dl in mice treated with tamoxifen, after 21 days after splenic injection of KPC cells. Taken together, the results from the evaluation of gene differential expression and insulin and glucose concentrations from Luminex measurements demonstrated that, even while insulin and blood glucose levels were approximately the same for mice treated with either diluent or TAM, Scd1 gene was highly expressed across all four mouse tissue types (“CHOP βHet,” “CHOP βKO,” “Tumor_WT,” and “Tumor_KO”).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A method of treating pancreatic cancer in a subject in need thereof, comprising:
inhibiting C/EBP homologous protein (CHOP) in pancreatic β cells by administering to the subject a composition comprising:
(a) a CHOP inhibiting moiety, and
(b) a pancreatic β cell targeting moiety.

2. A method of treating pancreatic cancer by regulating C/EBP homologous protein (CHOP) in pancreatic β cells, the method comprising administering a nucleic acid composition comprising:
(a) a CHOP inhibiting moiety, and
(b) a pancreatic β cell targeting moiety that directs the CHOP inhibiting moiety to its target in a pancreatic cell.

3. The method of claim 1, wherein the CHOP inhibiting moiety is a nucleic acid.

4. The method of claim 1, wherein the CHOP inhibiting moiety and the pancreatic β cell targeting moiety are operably linked.

5. (canceled)

6. (canceled)

7. (canceled)

8. (canceled)

9. The method of claim 1, wherein the pancreatic β cell targeting moiety is a peptide.

10. The method of claim 9, wherein the peptide is internalized by a pancreatic cell.

11. The method of claim 9, wherein the peptide is glucagon-like peptide 1 (GLP-1), or a fragment thereof.

12. The method of claim 1, wherein the CHOP inhibiting moiety is a nucleic acid editing moiety.

13. The method of claim 12, wherein the nucleic acid editing moiety is a genomic DNA editing moiety.

14. (canceled)

15. (canceled)

16. The method of claim 1, wherein the pancreatic β cell targeting moiety comprises a guiding nucleic acid sequence.

17. The method of claim 16, wherein the CHOP inhibiting moiety and/or the pancreatic β cell targeting moiety is inducible by an inducer.

18. (canceled)

19. (canceled)

20. The method of claim 1, wherein the subject is a mammal.

21. (canceled)

22. (canceled)

23. The method of claim 1, wherein the administering comprises administering to a subject systemically.

24. The method of claim 1, wherein the administering reduces or alleviates pancreatic β cell ER stress.

25.-60. (canceled)

61. A method of inhibiting pancreatic adenocarcinoma (PDAC) growth in a subject comprising, administering a treatment comprising a pharmaceutical composition wherein the pharmaceutical composition comprises:
(a) a CHOP inhibiting moiety, and
(b) a pancreatic β cell targeting moiety, and wherein the administering of the treatment inhibits C/EBP homologous protein (CHOP) in pancreatic β cell and further, wherein the inhibition of CHOP alleviates effects of β cell dysfunction.

62. (canceled)

63. The method of claim 61, wherein the effects of β cell dysfunction comprise initiation and progression of PDAC.

64. The method of claim 2, wherein the CHOP inhibiting moiety is a nucleic acid.

65. The method of claim 2, wherein the CHOP inhibiting moiety and the pancreatic 3 cell targeting moiety are operably linked.

66. The method of claim 2, wherein the pancreatic β cell targeting moiety is a peptide.

67. The method of claim 66, wherein the peptide is internalized by a pancreatic cell.


SEQUENCE LISTING (23 sequences)

SEQ ID NO: 1 - DNA, 28 bp, synthetic construct
  source 1..28: mol_type = other DNA
ctaacctattcctggtaagtggtatccg

SEQ ID NO: 2 - DNA, 42 bp, synthetic construct
  source 1..42: mol_type = other DNA
taagcattggtaagacgtcaagcccctctgaccttgtattac

SEQ ID NO: 3 - DNA, 43 bp, synthetic construct
  source 1..43: mol_type = other DNA
taagcattggtaagcggccgcgtgcactgatctgctagagctg

SEQ ID NO: 4 - DNA, 28 bp, synthetic construct
  source 1..28: mol_type = other DNA
ctaatgaacacagaaggggaggtttatg

SEQ ID NO: 5 - DNA, 20 bp, synthetic construct
  source 1..20: mol_type = other DNA
gaggggcttacaatgctttg

SEQ ID NO: 6 - DNA, 21 bp, synthetic construct
  source 1..21: mol_type = other DNA
gggtcgttcacctagagtaag

SEQ ID NO: 7 - DNA, 24 bp, synthetic construct
  source 1..24: mol_type = other DNA
aagagcagtagcacccagtgagtt

SEQ ID NO: 8 - DNA, 18 bp, synthetic construct
  source 1..18: mol_type = other DNA
agatcagtgcacctacaa

SEQ ID NO: 9 - DNA, 18 bp, synthetic construct
  source 1..18: mol_type = other DNA
caggatgcggacattgaa

SEQ ID NO: 10 - DNA, 25 bp, synthetic construct
  source 1..25: mol_type = other DNA
agcaaactctatggaagtggaggcc

SEQ ID NO: 11 - DNA, 23 bp, synthetic construct
  source 1..23: mol_type = other DNA
cttcttgacgagttcttctgagg

SEQ ID NO: 12 - DNA, 18 bp, synthetic construct
  source 1..18: mol_type = other DNA
aacaacagatggctggca

SEQ ID NO: 13 - DNA, 24 bp, synthetic construct
  source 1..24: mol_type = other DNA
tcagcctcgactgtgccttctagt

SEQ ID NO: 14 - DNA, 22 bp, synthetic construct
  source 1..22: mol_type = other DNA
attgaggagtggcagcatatag

SEQ ID NO: 15 - DNA, 24 bp, synthetic construct
  source 1..24: mol_type = other DNA
ggtagtctagtacctcctgtgata

SEQ ID NO: 16 - DNA, 26 bp, synthetic construct
  source 1..26: mol_type = other DNA
tgcttccttcagcactaccctttagc

SEQ ID NO: 17 - DNA, 21 bp, synthetic construct
  source 1..21: mol_type = other DNA
gattcagtagaccgctgttgg

SEQ ID NO: 18 - DNA, 22 bp, synthetic construct
  source 1..22: mol_type = other DNA
gcactgggatccctacattaac

SEQ ID NO: 19 - DNA, 28 bp, synthetic construct
  source 1..28: mol_type = other DNA
tgaaattaagagcagtagcacccagtga

SEQ ID NO: 20 - DNA, 18 bp, synthetic construct
  source 1..18: mol_type = other DNA
tatcctcccagggaaaga

SEQ ID NO: 21 - DNA, 18 bp, synthetic construct
  source 1..18: mol_type = other DNA
agcattgttctagaccgc

SEQ ID NO: 22 - DNA, 23 bp, synthetic construct
  source 1..23: mol_type = other DNA
aagcccctctgaccttgtattac

SEQ ID NO: 23 - protein, 4 aa, unidentified
KDEL